(19) United States
(12) Patent Application Publication
(10) Pub. No.: US 2026/0102483 A1
(43) Pub. Date: Apr. 16, 2026

(54) SUPERANTIGEN VACCINE CONJUGATE FOR THE TREATMENT OF CANCER

(71) Applicant: MUSC FOUNDATION FOR RESEARCH DEVELOPMENT, Charleston, NC (US)

(72) Inventors: Nathan G. DOLLOFF, Charleston, NC (US); Reeder M. ROBINSON, Charleston (UC); Lena GOLICK, Charleston, NC (US)

(73) Assignee: MUSC FOUNDATION FOR RESEARCH DEVELOPMENT, Charleston, NC (US)

(21) Appl. No.: 19/117,242

(22) PCT Filed: Oct. 4, 2023
(86) PCT No.: PCT/US2023/075949

§ 371 Date: Mar. 31, 2025

Related U.S. Application Data
(60) Provisional application No. 63/413,457, filed on Oct. 5, 2022.
(60) Provisional application No. 63/413,468, filed on Oct. 5, 2022.

(51) Int. Cl. A61K 39/385; A61K 31/7088; A61K 39/00; A61K 39/395; A61P 37/04

(52) U.S. Cl. CPC A61K 39/385; A61K 31/7088; A61K 39/001111; A61K 39/001126; A61K 39/001154; A61K 39/3955; A61P 37/04; A61K 2039/54; A61K 2039/6037

(57) ABSTRACT

The present disclosure provides compositions comprising vaccine conjugates with a SMEZ-2 carrier. Further provided are methods for treating cancer comprising administering the vaccine conjugates provided herein.

### PRIORITY CLAIM

This application claims benefit of priority to U.S. Provisional Application Ser. Nos. 63/413,457 and 63/413,468, filed Oct. 5, 2022, the entire contents of both of which are hereby incorporated by reference.

### BACKGROUND

### 1. Field

The disclosure relates generally to the field of molecular biology. More particularly, it concerns compositions comprising vaccine conjugates for the treatment of cancer.

### 2. Related Art

Pancreatic Ductal Adenocarcinoma (PDAC) accounted for 56,700 new cancer cases and 45,700 deaths in 2019 along with the lowest 5-year survival rate of all cancers at only 9% (Siegel et al., 2019). Treatment options are currently limited and have remained mostly unchanged with surgery, chemotherapy, gemcitabine, or FOLFIRINOX as the primary treatment options (Oettle et al., 2013; Oettle et al., 2007; Conroy et al., 2011; Conroy et al., 2018). Vaccine therapy has been used in PDAC patients in clinical trials previously, although with very limited success. Phase I/II trials with a synthetic KRAS-derived peptide showed detectable immune responses when given with GM-CSF and is currently in a clinical trial with checkpoint inhibition (NCT04117087) (Gjertsen et al., 1995; Gjertsen et al., 2001). A 16-aa peptide vaccine of telomerase (GV1001) that was designed to bind to multiple MHC molecules showed apparent efficacy in Phase I/II, but the Phase III PrimoVax and TeloVac trials showed no survival benefit (Bernhardt et al., 2006; Gunturu et al., 2013). Also, clinical trials of both a gastrin-based vaccine (G17DT) and a poxvirus-based vaccine encoding carcinoembryonic antigen and mucin 1 (PANVAC-V) showed no improvements in survival (Gilliam et al., 2012; Arlen et al., 2007)). GVAX® is an allogenic, GM-CSF expressing irradiated pancreatic cancer cell line vaccine currently in clinical trials (with checkpoint inhibition) with the attenuated, mesothelin expressing Listeria-based CRS-207 which are given in a prime/boost format (NCT03190265) (Tsujikawa et al., 2020). However, the Phase IIb ECLIPSE study with this vaccine combination alone showed no survival advantage over chemotherapy (Le et al., 2019). In addition to these overall failures in vaccine therapy against PDAC, objective responses with other immunotherapies have been largely absent. Thus, the lack of response in PDAC to existing immunotherapy options presents a need for new treatment options that would overcome these barriers.

The use of cancer vaccines to treat various malignancies in patients through targeted immune stimulation is appealing, although objective clinical responses are mostly limited (Rosenberg et al., 2004). While the lack of response is a complex issue, it has been primarily attributed to thymic and T cell tolerance towards self-antigens upregulated in various cancers (Melief et al., 2015). Neoantigens and tumor-associated antigens (TAAs) have been focused upon recently to generate cancer vaccines as their expression is typically restricted or absent in non-malignant cells, but aberrantly or over-expressed in cancer cells (Romero et al., 2016). In silico methods are commonly used to predict peptide epitopes of either TAAs or neoantigens that are both immunogenic and bind effectively to MHC molecules for recognition by T cells (Nat Biotechnol., 2017). These methods are still mostly theoretical, and with the complexity of multiple factors (peptide processing prediction, epitope binding to MHC molecules, mutation orientation towards T-cells, different MHC alleles in patients, etc.) there is a wide chasm to bridge for these software-based algorithms to have clinical usefulness.

Currently, sipuleucel-T is the only approved cancer vaccine and is used for the treatment of metastatic castration-resistant prostate cancer. Sipuleucel-T is an autologous dendritic cell vaccine and is prepared by extracting peripheral blood mononuclear leucocytes from a patient through leukapheresis, sending them to a production facility, and pulsing with several rounds of a recombinant fusion protein (prostatic acid phosphatase (PAP) and granulocyte-macrophage colony stimulating factor (GM-CSF)) to generate mature antigen presenting cells (APCs). These activated cells are then reinfused into patients to generate an immune response. Overall, sipuleucel-T was shown to improve median survival by 4.1 months but is a complicated and expensive procedure with significant manufacturing hurdles (Kantoff et al., 2010, Hammerstrom et al., 2011). To date, numerous other cancer vaccine strategies have been used in different cancer types but have generally been unsuccessful (Melero et al., 2014). While recent Phase I data from an RNA vaccine candidate in melanoma combined with checkpoint inhibition showed promising results breathing life back into the clinical usefulness of vaccinating against TAAs (Sahin et al., 2020), there is an unmet need for cancer vaccines for the treatment of cancers, such as PDAC.

While considerable progress has been made in the treatment of pediatric acute lymphoblastic leukemia (pALL), effective therapeutics for pediatric acute myeloid leukemia (pAML) have lagged behind. pAML is rarer than pALL, making up ˜20% of pediatric leukemia cases (Morais et al., 2020). However, it is also more deadly, with a five-year survival rate of just 67-68% (Siegel et al., 2022). One of the challenges in treating pAML is the heterogeneity of the disease. The French-American-British classification of AML divides the disease into eight subtypes, M0-M7. Each subtype is characterized by a specific hematopoietic lineage and stage of maturity. While adult AML (aAML) is thought to be caused by an accumulation of somatic mutations, these mutations are relatively rare in pAML. It is much more common for pediatric patients to exhibit cytogenetic abnormalities that lead to de novo AML (Bolouri et al., 2017). These features make it challenging to develop a targeted therapeutic that works for most patients. Furthermore, the predominant focus for AML treatments has been for aAML, with the hope that they will also work for pAML. However, the differences between aAML and pAML suggest that a therapy may be effective for one, but not the other.

Presently, one CD33-targeted therapy is FDA approved for pAML-gemtuzumab ozogamicin (GO), an antibody-drug conjugate (ADC). GO is comprised of an anti-CD33 monoclonal antibody linked to a cytotoxic calicheamicin payload. Upon endocytosis of the ADC, the linker is cleaved and calicheamicin induces cell death through double-strand DNA breaks. However, there are multiple disadvantages to GO that suggest it may not be the best method for targeting CD33. One major drawback is that the binding epitope for GO is on the Immunoglobulin-like V (IgV) domain. 50% of CD33-positive AML patients express a single nucleotide polymorphism (SNP), rs12459419 C>T, that leads to a CD33 splice variant lacking the IgV domain (Lamba et al., 2017). This renders GO ineffective in half of the patients eligible for its use because the CD33 protein they express lacks the gemtuzumab binding epitope.

Furthermore, premature cleavage of the linker between gemtuzumab and the calicheamicin payload can cause off-target effects. Another ADC, inotuzumab ozogamicin, is a CD22 targeted agent that also utilizes calicheamicin. It has been shown that the usage of these drugs can increase the risk of venoocclusive disease (VOD) in patients that receive hematopoietic stem cell transplants (HSCTs) (Ladha et al., 2020). Another potential issue with GO is resistance to calicheamicin. Previous studies have shown that the expression of P-glycoprotein (Pgp) on AML blasts can lead to drug efflux and reduce the efficacy of GO (Linenberger et al., 2001).

Lastly, GO has only shown a modest improvement in a clinical trial comparing pAML patients receiving standard chemotherapy vs. chemotherapy with the addition of GO. Event free survival (EFS) was significantly improved for patients receiving GO vs. standard chemotherapy, but there was no difference in overall survival (OS). When patients were stratified into low-risk, intermediate-risk, and high-risk groups, none showed a significant increase in event free survival (Gamis et al., 2014). Therefore, there is still a significant need for novel pAML treatments and CD33 has yet to reach its maximum potential as a target.

### SUMMARY

In a first embodiment, the present disclosure provides vaccine conjugates comprising mutant Streptococcal Mitogenic Exotoxin Z-2 (SMEZ-2) conjugated to at least one target protein or fragment thereof, wherein the at least one target protein is overexpressed in a cancer.

In some aspects, the mutant SMEZ-2 comprises mutations W75L and K182Q. In certain aspects, the mutant SMEZ-2 comprises mutations W75L, K182Q, and/or D42C. In particular aspects, the mutant SMEZ-2 comprises mutations W75L, K182Q, and D42C.

In some aspects, the conjugate comprises a linker, such as between SMEZ-2 and the at least one target protein. In certain aspects, the linker is a peptide linker, such as a glycine-serine linker. For example, the linker is AIA or GGGGS.

In certain aspects, the at least one target protein is a neoantigen or tumor-associated antigen (TAA). In specific aspects, the target protein is anterior gradient 2 (AGR2), such as AGR2 is human AGR2. In some aspects, the at least one target protein is human epidermal growth factor receptor 2 (HER2), prostate-specific membrane antigen (PSMA), melanoma-associated antigen 3 (MAGE-A3). NY-ESO-1, IL-8, or GDF-15. In some aspects, the at least one target protein is CD33 or a fragment thereof, mesothelin (MSLN), B-cell maturation antigen (BCMA), GPRC5D, CD123. CLL-1 (CD371), CD19, CD30, or CD20. In some aspects, the CD33 or fragment thereof comprises one or more amino acid substitutions. In some aspects, full length CD33 has an amino acid substitution at Asparagine 98. In some aspects, CD33 comprises a truncated version of CD33 consisting of the CD33-IgC domain, such as CD33-IgC comprising an amino acid substitution at Aspartic acid 231 or Cysteine 154. In some aspects, the one or more amino acid substitutions are at D231, D246. C154 and/or C169. In some aspects, the one or more amino acid substitutions are D231E, D246E, C154S and/or C169S.

In certain aspects, the vaccine conjugate comprises at least a second target protein or fragment thereof. In some aspects, the at least one target protein is CD38.

In some aspects, the at least one target protein is an immune checkpoint protein. In particular aspects, the immune checkpoint protein is CTLA-4, PD-1, PD-L1, PD-L2, LAG-3, BTLA, B7H3, B7H4, TIM3, KIR, or A2aR.

A further embodiment provides a pharmaceutical composition comprising a vaccine conjugate of the present embodiments (e.g., vaccine conjugates comprising mutant Streptococcal Mitogenic Exotoxin Z-2 (SMEZ-2) conjugated to at least one target protein or fragment thereof, wherein the at least one target protein is overexpressed in a cancer) and an adjuvant. In some aspects, the adjuvant is Incomplete Freund's Adjuvant. In certain aspects, the conjugate is formulated as a 1:1 emulsification with Incomplete Freund's Adjuvant (IFA). In some aspects, the conjugate is formulated for intravenous infusion, subcutaneous injection, or intramuscular injection.

Another embodiment provides an expression vector comprising a sequence encoding mutant SMEZ-2 fused to a sequence encoding a target protein or fragment thereof. In some aspects, the target protein is a neoantigen or TAA. In certain aspects, the vector encodes a vaccine conjugate of the present embodiments (e.g., vaccine conjugates comprising mutant Streptococcal Mitogenic Exotoxin Z-2 (SMEZ-2) conjugated to at least one target protein or fragment thereof). Also provided herein is a host cell comprising an expression vector of the present embodiments, such as E. coli, human embryonic kidney cell (HEK293), or Chinese hamster ovary (CHO) cell.

In yet another embodiment, there is provided a method for stimulating an immune response in a subject, comprising administering to the subject an effective amount of a vaccine conjugate of the present embodiments (e.g., vaccine conjugates comprising mutant Streptococcal Mitogenic Exotoxin Z-2 (SMEZ-2) conjugated to at least one target protein or fragment thereof, wherein the at least one target protein is overexpressed in a cancer) or a pharmaceutical composition of the present embodiments.

In some aspects, the immune response is an anti-cancer immune response. In certain aspects, the subject has a cancer. In specific aspects, the cancer is oral cancer, oropharyngeal cancer, nasopharyngeal cancer, respiratory cancer, urogenital cancer, gastrointestinal cancer, central or peripheral nervous system tissue cancer, an endocrine or neuroendocrine cancer or hematopoietic cancer, glioma, sarcoma, carcinoma, lymphoma, melanoma, fibroma, meningioma, brain cancer, oropharyngeal cancer, nasopharyngeal cancer, renal cancer, biliary cancer, pheochromocytoma, pancreatic islet cell cancer. Li-Fraumeni tumors, thyroid cancer, parathyroid cancer, pituitary tumors, adrenal gland tumors, osteogenic sarcoma tumors, multiple neuroendocrine type I and type II tumors, breast cancer, lung cancer, head and neck cancer, prostate cancer, esophageal cancer, tracheal cancer, liver cancer, bladder cancer, stomach cancer, pancreatic cancer, ovarian cancer, uterine cancer, cervical cancer, testicular cancer, colon cancer, rectal cancer or skin cancer. In particular aspects, the cancer is pancreatic cancer, such as pancreatic ductal adenocarcinoma (PDAC). In certain aspects, the cancer is breast cancer. In some aspects, the cancer is multiple myeloma or lymphoma, such as T cell non-Hodgkin's lymphoma.

In certain aspects, the immune response is an anti-AGR2 specific immune response. In some aspects, the anti-AGR2 specific immune response is detected by measuring an increased titer of AGR2 specific immunoglobulins in a sample of said subject's blood. In other aspects, the immune response is an anti-CD38, anti-PD1, anti-CTLA4, or anti-PDL1 response and is measured by measuring an increased titer of CD38, PD1, CTLA4, or PDL1 specific immunoglobulins in a sample of said subject's blood.

In some aspects, the conjugate is administered by injection, such as intravenous infusion.

In additional aspects, the method further comprises administering a second anti-cancer therapy to said subject. In some aspects, the second anti-cancer therapy is immunotherapy, chemotherapy, radiotherapy, gene therapy, surgery, hormonal therapy, anti-angiogenic therapy or cytokine therapy. In some aspects, the second anti-cancer therapy is immunotherapy, such as an immune checkpoint inhibitor. For example, the immune checkpoint inhibitor is selected from an inhibitor of CTLA-4, PD-1, PD-L1, PD-L2, LAG-3, BTLA, B7H3, B7H4, TIM3, KIR, or A2aR. In specific aspects, the immune checkpoint inhibitor comprises an anti-PD1 agent, such as an anti-PD1 antibody, anti-PDL1 antibody, or an anti-PDL2 antibody. In certain aspects, the anti-PD1 agent is nivolumab, pembrolizumab, pidilizumab, KEYTRUDA®, AMP-514, REGN2810, CT-011, BMS 936559, MPDL328OA or AMP-224. In some aspects, the immune checkpoint inhibitor is an anti-CTLA-4 antibody. For example, the anti-CTLA-4 antibody is tremelimumab. YERVOY®, or ipilimumab. In some aspects, the further anti-cancer therapy comprises a TLR9 agonist, such as CpG ODN1826, and/or CD40 agonist, such as CD40 agonistic antibody. In certain aspects, the subject is administered an immune checkpoint inhibitor and TLR9 agonist.

A further embodiment provides a method of treating a subject having a cancer comprising administering a vaccine conjugate of the present embodiments (e.g., vaccine conjugates comprising mutant Streptococcal Mitogenic Exotoxin Z-2 (SMEZ-2) conjugated to at least one target protein or fragment thereof, wherein the at least one target protein is overexpressed in a cancer) or a pharmaceutical composition of the present embodiments to the subject.

In some aspects, the cancer is oral cancer, oropharyngeal cancer, nasopharyngeal cancer, respiratory cancer, urogenital cancer, gastrointestinal cancer, central or peripheral nervous system tissue cancer, an endocrine or neuroendocrine cancer or hematopoietic cancer, glioma, sarcoma, carcinoma, lymphoma, melanoma, fibroma, meningioma, brain cancer, oropharyngeal cancer, nasopharyngeal cancer, renal cancer, biliary cancer, pheochromocytoma, pancreatic islet cell cancer, Li-Fraumeni tumors, thyroid cancer, parathyroid cancer, pituitary tumors, adrenal gland tumors, osteogenic sarcoma tumors, multiple neuroendocrine type I and type II tumors, breast cancer, lung cancer, head and neck cancer, prostate cancer, esophageal cancer, tracheal cancer, liver cancer, bladder cancer, stomach cancer, pancreatic cancer, ovarian cancer, uterine cancer, cervical cancer, testicular cancer, colon cancer, rectal cancer or skin cancer. In specific aspects, the cancer is pancreatic cancer, such as pancreatic ductal adenocarcinoma (PDAC). In particular aspects, the cancer is breast cancer. In some aspects, the cancer is multiple myeloma or lymphoma, such as T cell non-Hodgkin's lymphoma.

In further aspects, the method further comprises administering at least one immune checkpoint inhibitor to the subject. In some aspects, administering at least one immune checkpoint inhibitor to the subject comprises administering at least one immune checkpoint inhibitor before the vaccine conjugate. In other aspects, administering at least one immune checkpoint inhibitor to the subject comprises administering at least one immune checkpoint inhibitor after or concomitantly with the vaccine conjugate. For example, the immune checkpoint inhibitor is selected from an inhibitor of CTLA-4, PD-1, PD-L1, PD-L2, LAG-3, BTLA, B7H3, B7H4, TIM3, KIR, or A2aR. In specific aspects, the immune checkpoint inhibitor comprises an anti-PD1 agent, such as an anti-PD1 antibody, anti-PDL1 antibody, or an anti-PDL2 antibody. In certain aspects, the anti-PD1/PDL agent is nivolumab, pembrolizumab, pidilizumab, KEYTRUDA®, AMP-514, REGN2810, CT-011, BMS 936559, MPDL328OA or AMP-224. In some aspects, the immune checkpoint inhibitor is an anti-CTLA-4 antibody. For example, the anti-CTLA-4 antibody is tremelimumab, YERVOY®, or ipilimumab. In some aspects, the subject is administered two immune checkpoint inhibitors. In particular aspects, the two immune checkpoint inhibitors are an anti-PD1 antibody and an anti-CTL4 antibody. In some aspects, the vaccine conjugate is administered two or more times. In some aspects, the further anti-cancer therapy comprises a TLR9 agonist, such as CpG ODN1826, and/or CD40 agonist, such as CD40 agonistic antibody. In certain aspects, the subject is administered an immune checkpoint inhibitor and TLR9 agonist.

In additional aspects, the method further comprises administering a further anti-cancer therapy to the subject. In some aspects, the further anti-cancer therapy is chemotherapy, radiotherapy, gene therapy, surgery, hormonal therapy, anti-angiogenic therapy or cytokine therapy.

Another embodiment provides a kit comprising a vaccine conjugate of the present embodiments (e.g., vaccine conjugates comprising mutant Streptococcal Mitogenic Exotoxin Z-2 (SMEZ-2) or a pharmaceutical composition of the present embodiments. In some aspects, the kit further comprises an immune checkpoint inhibitor. For example, the immune checkpoint inhibitor is an anti-PD1 antibody or CTLA-4 antibody.

A further embodiment provides a composition comprising a vaccine conjugate of the present embodiments (e.g., vaccine conjugates comprising mutant Streptococcal Mitogenic Exotoxin Z-2 (SMEZ-2) or a pharmaceutical composition of the present embodiments for use in treating a cancer. In some aspects, the composition further comprises an immune checkpoint inhibitor.

In another embodiment, the present disclosure provides vaccine conjugates comprising mutant Streptococcal Mitogenic Exotoxin Z-2 (SMEZ-2) conjugated to CD33 or fragment thereof.

In some aspects, the mutant SMEZ-2 comprises mutations W75L and K182Q. In certain aspects, the mutant SMEZ-2 comprises mutations W75L, K182Q, and/or D42C. In particular aspects, the mutant SMEZ-2 comprises mutations W75L. K182Q. and D42C. In certain aspects, the vaccine conjugate comprises at least a second target protein or fragment thereof.

In some aspects, the CD33 or fragment thereof comprises one or more amino acid substitutions. In some aspects, full length CD33 has an amino acid substitution at Asparagine 98. In some aspects, CD33 comprises a truncated version of CD33 consisting of the CD33-IgC domain, such as CD33-IgC comprising an amino acid substitution at Aspartic acid 231 or Cysteine 154. In some aspects, the one or more amino acid substitutions are at D231, D246, C154 and/or C169. In certain aspects, the one or more amino acid substitutions are D231E, D246E, C154S and/or C169S.

In certain aspects, the linker is a peptide linker, such as a glycine serine linker, particularly a GGGGS linker.

A further embodiment provides a pharmaceutical composition comprising a vaccine conjugate of the present embodiments (e.g., vaccine conjugates comprising mutant Streptococcal Mitogenic Exotoxin Z-2 (SMEZ-2) conjugated to CD33) and an adjuvant. In some aspects, the adjuvant is Incomplete Freund's Adjuvant. In certain aspects, the conjugate is formulated as a 1:1 emulsification with Incomplete Freund's Adjuvant (IFA). In some aspects, the conjugate is formulated for intravenous infusion, subcutaneous, or intramuscular injection.

Another embodiment provides an expression vector comprising a sequence encoding mutant SMEZ-2 fused to a sequence encoding CD33 or fragment thereof. Also provided herein is a host cell comprising an expression vector of the present embodiments, such as E. coli, human embryonic kidney cell (HEK293), or Chinese hamster ovary (CHO) cell.

In yet another embodiment, there is provided a method for stimulating an immune response in a subject, comprising administering to the subject an effective amount of a vaccine conjugate of the present embodiments (e.g., vaccine conjugates comprising mutant Streptococcal Mitogenic Exotoxin Z-2 (SMEZ-2) conjugated to CD33 or fragment thereof) or a pharmaceutical composition of the present embodiments.

In some aspects, the immune response is an anti-cancer immune response. In certain aspects, the subject has a cancer. In specific aspects, the cancer is oral cancer, oropharyngeal cancer, nasopharyngeal cancer, respiratory cancer, urogenital cancer, gastrointestinal cancer, central or peripheral nervous system tissue cancer, an endocrine or neuroendocrine cancer or hematopoietic cancer, glioma, sarcoma, carcinoma, lymphoma, melanoma, fibroma, meningioma, brain cancer, oropharyngeal cancer, nasopharyngeal cancer, renal cancer, biliary cancer, pheochromocytoma, pancreatic islet cell cancer, Li-Fraumeni tumors, thyroid cancer, parathyroid cancer, pituitary tumors, adrenal gland tumors, osteogenic sarcoma tumors, multiple neuroendocrine type I and type II tumors, breast cancer, lung cancer, head and neck cancer, prostate cancer, esophageal cancer, tracheal cancer, liver cancer, bladder cancer, stomach cancer, pancreatic cancer, ovarian cancer, uterine cancer, cervical cancer, testicular cancer, colon cancer, rectal cancer or skin cancer. In particular aspects, the cancer is acute myelogenous leukemia.

In certain aspects, the immune response is an anti-CD33 specific immune response. In some aspects, the anti-CD33 specific immune response is detected by measuring an increased titer of CD33 specific immunoglobulins in a sample of said subject's blood.

In some aspects, the conjugate is administered by injection, such as intravenous infusion.

In additional aspects, the method further comprises administering a second anti-cancer therapy to said subject. In some aspects, the second anti-cancer therapy is immunotherapy, chemotherapy, radiotherapy, gene therapy, surgery, hormonal therapy, anti-angiogenic therapy or cytokine therapy. In some aspects, the second anti-cancer therapy is immunotherapy, such as an immune checkpoint inhibitor. For example, the immune checkpoint inhibitor is selected from an inhibitor of CTLA-4, PD-1, PD-L1, PD-L2, LAG-3, BTLA, B7H3, B7H4, TIM3, KIR, or A2aR. In specific aspects, the immune checkpoint inhibitor comprises an anti-PD1 agent, such as an anti-PD1 antibody, anti-PDL1 antibody, or an anti-PDL2 antibody. In certain aspects, the anti-PD1 agent is nivolumab, pembrolizumab, pidilizumab, KEYTRUDA®, AMP-514, REGN2810, CT-011. BMS 936559. MPDL328OA or AMP-224. In some aspects, the immune checkpoint inhibitor is an anti-CTLA-4 antibody. For example, the anti-CTLA-4 antibody is tremelimumab. YERVOY®, or ipilimumab.

A further embodiment provides a method of treating a subject having a cancer comprising administering a vaccine conjugate of the present embodiments (e.g., vaccine conjugates comprising mutant Streptococcal Mitogenic Exotoxin Z-2 (SMEZ-2) conjugated to CD33 or fragment thereof) or a pharmaceutical composition of the present embodiments to the subject.

In some aspects, the cancer is oral cancer, oropharyngeal cancer, nasopharyngeal cancer, respiratory cancer, urogenital cancer, gastrointestinal cancer, central or peripheral nervous system tissue cancer, an endocrine or neuroendocrine cancer or hematopoietic cancer, glioma, sarcoma, carcinoma, lymphoma, melanoma, fibroma, meningioma, brain cancer, oropharyngeal cancer, nasopharyngeal cancer, renal cancer, biliary cancer, pheochromocytoma, pancreatic islet cell cancer, Li-Fraumeni tumors, thyroid cancer, parathyroid cancer, pituitary tumors, adrenal gland tumors, osteogenic sarcoma tumors, multiple neuroendocrine type I and type II tumors, breast cancer, lung cancer, head and neck cancer, prostate cancer, esophageal cancer, tracheal cancer, liver cancer, bladder cancer, stomach cancer, pancreatic cancer, ovarian cancer, uterine cancer, cervical cancer, testicular cancer, colon cancer, rectal cancer or skin cancer. In specific aspects, the cancer is acute myelogenous leukemia.

In further aspects, the method further comprises administering at least one immune checkpoint inhibitor to the subject. In some aspects, administering at least one immune checkpoint inhibitor to the subject comprises administering at least one immune checkpoint inhibitor before the vaccine conjugate. In other aspects, administering at least one immune checkpoint inhibitor to the subject comprises administering at least one immune checkpoint inhibitor after or concomitantly with the vaccine conjugate. For example, the immune checkpoint inhibitor is selected from an inhibitor of CTLA-4, PD-1, PD-L1, PD-L2, LAG-3, BTLA, B7H3, B7H4, TIM3, KIR, or A2aR. In specific aspects, the immune checkpoint inhibitor comprises an anti-PD1/PDL agent, such as an anti-PD1 antibody, anti-PDL1 antibody, or an anti-PDL2 antibody. In certain aspects, the anti-PD1 agent is nivolumab, pembrolizumab, pidilizumab, KEYTRUDA®, AMP-514, REGN2810, CT-011, BMS 936559, MPDL328OA or AMP-224. In some aspects, the immune checkpoint inhibitor is an anti-CTLA-4 antibody. For example, the anti-CTLA-4 antibody is tremelimumab, YERVOY®, or ipilimumab. In some aspects, the subject is administered two immune checkpoint inhibitors. In particular aspects, the two immune checkpoint inhibitors are an anti-PD1 antibody and an anti-CTL4 antibody. In some aspects, the vaccine conjugate is administered two or more times.

In additional aspects, the method further comprises administering a further anti-cancer therapy to the subject. In some aspects, the further anti-cancer therapy is chemotherapy, radiotherapy, gene therapy, surgery, hormonal therapy, anti-angiogenic therapy or cytokine therapy.

Another embodiment provides a kit comprising a vaccine conjugate of the present embodiments (e.g., vaccine conjugates comprising mutant Streptococcal Mitogenic Exotoxin Z-2 (SMEZ-2)) or a pharmaceutical composition of the present embodiments. In some aspects, the kit further comprises an immune checkpoint inhibitor. For example, the immune checkpoint inhibitor is an anti-PD1 antibody or CTLA-4 antibody.

A further embodiment provides a composition comprising a vaccine conjugate of the present embodiments (e.g., vaccine conjugates comprising mutant Streptococcal Mitogenic Exotoxin Z-2 (SMEZ-2) or a pharmaceutical composition of the present embodiments for use in treating a cancer. In some aspects, the composition further comprises an immune checkpoint inhibitor.

It is contemplated that any method or composition described herein can be implemented with respect to any other method or composition described herein. For example, a compound synthesized by one method may be used in the preparation of a final compound according to a different method.

The use of the word “a” or “an” when used in conjunction with the term “comprising” in the claims and/or the specification may mean “one,” but it is also consistent with the meaning of “one or more,” “at least one,” and “one or more than one.” The word “about” means plus or minus 5% of the stated number.

Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1: Mechanism of antigen presentation facilitated by mutant SMEZ-2.

FIG. 2: SDS-PAGE of purified proteins. (1) Molecular Weight Marker (2) AGR2 (3) SMEZ-2 (W75L, K182Q, D42C) (4) AGR2-SMEZ-2 (W75L, K182Q, D42C).

FIG. 3: Engineered construct of AGR2-SMEZ-2 conjugate expressed in E. coli.

FIG. 4: ELISA data of anti-AGR2 IgG in blood plasma of C57BL/6 mice vaccinated with 100 μg of AGR2-SMEZ2 or equimolar amounts of the indicated controls. Proteins were administered by intramuscular injection.

FIGS. 5A-5B: C57BL/6 mice were treated according to the schedule shown in FIG. 5A. Mice were injected with 100 μg AGR2-SMEZ or an equimolar amount of AGR2 (FIG. 5A), 50 μg of ODN 1826 (CpG. C), and implanted with 100,000 AGR2-expressing KPC cells (T). Proteins were administered subcutaneously in IFA. ODN1826 was given by intramuscular injection. (FIG. 5B) Tumor volumes were measured 16 days after implantation.

FIGS. 6A-6B: Recombinant mouse CD38, SMEZ2, and CD38-SMEZ2 conjugate were expressed and purified from mammalian cells. (FIG. 6A) SDS-PAGE analysis is shown. (FIG. 6B) Blood plasma was drawn from Balb/c mice 10 days after two injections of 50 μg mCD38-SMEZ subQ in IFA (or equimolar equivalents of controls). Serum was diluted 1:10,000 and anti-CD38 IgG were measured by ELISA.

FIGS. 7A-7C: SDS-PAGE of purified proteins. (FIG. 7A) Mouse CTLA-4 and CTLA-4 SMEZ. (FIG. 7B) Mouse PD-1 and mouse PD1-SMEZ (FIG. 7C) mouse PD-L1 and PDL1-SMEZ.

FIGS. 8A-8B: In vivo activity of SMEZ-PD1, SMEZ-PD-L1, and SMEZ-CTLA4. (FIG. 8A) C57BL/6 mice were given 3 injections of a cocktail consisting of SMEZ conjugates of mouse PD-1, PDL1, and CTLA-4. Blood was drawn and IgG specific for each antigen were quantified by ELISA. (FIG. 8B) Splenocytes were harvested from mice in FIG. 8A and analyzed by flow cytometry for CD4+ and CD8+ T cells. Both populations of T cells were significantly reduced by the SMEZ cocktail.

FIGS. 9A-9C: CD33-SMEZ-2 preclinical efficacy in a mouse model of AML. (FIG. 9A) The indicated proteins were expressed and purified from Expi293 HEK cells. Western blots are shown for each of the proteins which express a 6×His tag for affinity purification. (FIG. 9B) C57BL/6 mice were injected subcutaneously with 40 pmol of the indicated protein in an IFA emulsification. After 3 doses, blood was drawn and ELISA assays were performed to detect anti-CD33 specific IgG in the blood plasma of inoculated mice. ELISA data are shown. (FIG. 9C) Two weeks after treatment of C57BL/6 mice with CD33-SMEZ-2, mice were challenged with 1×10⁶ C1498-hCD33 cells by intravenous injection. Survival data are shown.

FIGS. 10A-10C: (FIG. 10A) C57BL/6 mice were orthotopically injected with 15,000 KPC cells that were derived from a syngeneic pancreas tumor originating in LSL-Kras^{G12D}; LSL-Trp53^{R172H}; PdX1-cre mice which overexpress hAGR2. KPC cells were injected into the head of the pancreas on Day 0. Mice were treated with 50 μg SMEZ-AGR2 or SMEZ-MSLN (mesothelin) (subQ; emulsified in IFA) twice prior to orthotopic injection and twice after (Days: −24, −10, 4, and 18). Mice were treated with 50 μg ODN 1826 (IM) and 250 μg α-mPD-1 (IM; BioXCell; clone RMP1-14) 3×/week for 3 weeks starting on Day 3 (Days: 3, 5, 7, 10, 12, 14, 17, 19, 21). Control mice received vehicle for all 3 treatments. Mice were sacrificed on Day 25 and tumors were excised. (FIG. 10B) photographed, and (FIG. 10C) weighed.

FIGS. 11A-11C: (FIG. 11A) Protein structures of various CD33-SMEZ constructs are shown. The upper panel shows full length CD33-SMEZ, which contains the two extracellular domains of CD33. IgV and IgC, connected to modified SMEZ-2 superantigen. The bottom left model depicts CD33 IgC-SMEZ, containing only the IgC domain of CD33 conjugated to modified SMEZ-2. Lastly, the bottom right shows the CD33 IgC-SMEZ-3Mut, an optimized version of CD33 IgC-SMEZ with three amino acid substitutions that we hypothesized would stabilize the protein, reduce aggregation, and increase the developability of the protein. (FIG. 11B) Western blots (left panel) show the original and versions of CD33 IgC and CD33 IgC-SMEZ under non-reducing conditions. Proteins were visualized by blotting for the 6×His affinity tag that was engineered into all proteins. Both CD33 IgC and CD33 IgC-SMEZ exhibit aggregation of the proteins that is diminished in the 3Mut candidate upon introduction of the three amino acid mutations. (Right panel) Protein aggregation was quantified by Proteostat protein aggregation assay, and confirmed significant reduction in aggregation for both CD33 IgC and CD33 IgC-SMEZ (referred to here as M2T) compared to the 3Mut versions of the proteins. (FIG. 11C) C57BL/6 mice were treated with 10 ug or 2 ug of CD33 IgC-SMEZ, equimolar doses of CD33 IgC alone, or vehicle control (PBS) intramuscularly. One week after the fourth dose, α-CD33 antibody titers from the blood plasma of mice were measured by enzyme linked immunosorbent assay (ELISA). As expected, the SMEZ conjugated protein induced high levels of anti-IgC IgG in mice compared to the IgC protein alone.

### DESCRIPTION OF ILLUSTRATIVE EMBODIMENTS

Anterior Gradient 2 (AGR2) is a member of the protein disulfide isomerase (PDI) family that is involved in forming disulfide bonds for newly synthesized proteins in the endoplasmic reticulum (ER). Like other proteins in the PDI family, AGR2 possesses an N-terminal ER leader sequence and a C-terminal ER retention signal (Patel et al., 2013). Despite these sequence properties that localize and retain AGR2 to the ER, it is also observed extracellularly where it interacts with the extracellular matrix and is linked to increased invasiveness, proliferation, survival, and metastasis in several human cancers, including PDAC (Fessart et al., 2016; Moidu et al., 2020; Di Maro et al., 2014; Ma et al., 2017; Wang et al., 2008). Indeed, significantly increased extracellular AGR2 was observed in both immunohistochemical (IHC) staining and tissue microarray of PDAC, but not in normal pancreatic tissue from patient samples. Consequently, the elevated expression levels of AGR2 along with its pro-oncogenic features led to the hypothesis that AGR2 is an enticing TAA immunotherapy target in PDAC.

Thus, in certain embodiments, vaccine conjugates are provided herein, such as a detoxified AGR2-SMEZ-2 conjugate that can stimulate a robust immune response against PDAC expressing high AGR2. This conjugate harnesses the MHC II binding abilities of SMEZ-2 to allow for efficient uptake and presentation of AGR2 peptides by APCs. This conjugate can lead to a robust anti-PDAC immune response and tumor eradication.

This vaccine approach provides multiple advantages to monoclonal antibody (mAb) therapy, which include adaptability to other tumor types and conjugation with different TAAs, lower manufacturing costs, and greater durability. Also, the elicited polyclonal response may be less likely to lead to therapeutic resistance, as single AGR2 mutations are unlikely to escape the immune response. This technology overcomes current challenges presented by peptide-based cancer vaccines where in silico simulations with peptide binding to MHC molecules are no longer necessary due to the high affinity binding properties of SMEZ-2. Therefore, further embodiments provide vaccine conjugates for other TAAs and neoantigen-based vaccine candidates.

In particular aspects, bacterial superantigen conjugates are provided herein by cloning tumor associated antigens or immune targets (i.e., AGR2. CD38, PD-1, PD-L1, CTLA-4) in cis to a mutant version of the SMEZ-2 bacterial superantigen. The mutant superantigen has high affinity for MHC class II molecules thereby facilitating and enhancing the presentation of antigens to immune effector cells and stimulating an immune response against the target cells expressing the antigen linked to SMEZ2. The present data showed that the AGR2-SMEZ2, CD38-SMEZ, PD-1-SMEZ, PDL1-SMEZ, and CTLA4-SMEZ conjugates induce robust humoral responses with high titers of specific immunoglobulins detected in the blood plasma of treated mice.

Sialic acid binding Ig-like lectin 3 (CD33) is an attractive therapeutic target for acute myeloid leukemia (AML). It is a member of the Siglec family, characterized by cell-cell interactions through binding of sialylated glycans. CD33 consists of two extracellular domains, an Immunoglobulin-like V (IgV) domain and an Immunoglobulin-like C (IgC) domain, a transmembrane domain, an immunoreceptor tyrosine-based inhibitory motif (ITIM) domain, and an ITIM-like domain. Although the signaling function and role of CD33 is not well understood, it is thought to inhibit cellular activation and proliferation upon ligand binding. CD33 is normally expressed on myeloid cells and is present in ˜85-90% of both pAML and aAML cases. It is notably not expressed on lymphocytes or erythrocytes.

Streptococcal mitogenic exotoxin Z-2 (SMEZ-2) is a streptococcal superantigen (SAg) that cross-links MHC class II and the T cell receptor (TCR), leading to mature T cell proliferation, overproduction of cytokines, and eventually T cell anergy (Deacy et al., 2021). Radcliff et al. previously showed that a modified version, called SMEZ-2 M1, can prevent TCR binding, eliminating the subsequent cytokine storm. SMEZ-2 M1 preserves the ability to bind MHC class II and can enhance presentation of conjugated antigens to the immune system (Radcliff et al., 2012).

Thus, in certain embodiments, vaccine conjugates are provided herein, such as a detoxified CD33-SMEZ-2 conjugate. In the present studies, the inventors developed a CD33 targeted immunotherapy by cloning human CD33 linked to a mutant version of the SMEZ-2 bacterial superantigen. The mutant superantigen had high affinity for MHC class II molecules thereby facilitating and enhancing the presentation of CD33 peptides to immune effector cells and stimulating an anti-CD33 specific immune response against AML. The data showed that the CD33-SMEZ-2 conjugate induced a robust anti-CD33 humoral response with high titers of CD33 specific immunoglobulins detected in the blood plasma of treated mice. Additional animal studies demonstrated that these effects were sufficient to reduce AML burden in mice and significantly prolong survival.

Another embodiment provides a CD33 vaccine conjugate of the present embodiments (e.g., vaccine conjugate with mutant Streptococcal Mitogenic Exotoxin Z-2 (SMEZ-2)) wherein strategic amino acid substitutions have been made to the full length or truncated CD33 protein. In some aspects, full length CD33 was modified at predicted sites of deamidation, including Asparagine 98 to improve the stability and prevent protein aggregation and to overall improve drug suitability of the candidate. In some aspects, a truncated version of CD33 consisting only of the CD33-IgC domain was further modified by mutation of amino acids predicted to be high risk sites for isomerization, including Aspartic acid 231, or free cysteines, including Cysteine 154 to improve the stability and prevent protein aggregation and to overall improve drug suitability of the candidate.

### I. CANCER VACCINE CONJUGATES

The present compositions and methods can comprise cancer vaccines as a form of active immunotherapy where an antigenic peptide, polypeptide or protein or an autologous or allogenic tumor cell composition or “vaccine” is administered to a subject. Vaccines may be administered systemically, such as intravenously, intradermally, or by intramuscular injection. Vaccines may also be administered multiple times to enhance the immune response against the administered antigens.

The term “vaccine” is used according to its plain ordinary meaning within medicine and Immunology and refers to a composition including an antigenic component (e.g., antigenic protein) for administration to a subject (e.g., human), which elicits an immune response to the antigenic component (e.g., antigenic protein). In some embodiments, a vaccine is a therapeutic. In some embodiments, a vaccine is prophylactic. In some embodiments a vaccine includes one or more adjuvants (e.g., aluminum adjuvant). A liquid vaccine is a vaccine in liquid form, which may be for example a solution, suspension, emulsion, or dispersion or the antigenic component (e.g., antigenic protein) of the vaccine and may optionally include other components. A dry vaccine is a vaccine comprising 5% or less of water.

A vaccine is a preparation employed to improve immunity to a particular disease. Vaccines include an agent, which is used to induce a response from the immune system of the subject. Various agents that are typically used in a vaccine include, but are not limited to: killed, but previously virulent, micro-organisms; live, attenuated microorganisms; inactivated toxic compounds that are produced by microorganism that cause an illness; protein subunits of microorganisms; and conjugates.

The term “prime-boost” or “prime boost” as applied to a methodology of administering vaccines is used according to its plain ordinary meaning in Virology and Immunology and refers to a method of vaccine administration in which a first dose of a vaccine or vaccine component is administered to a subject or patient to begin the administration (prime) and at a later time (e.g., hours, days, weeks, months later) a second vaccine is administered to the same patient or subject (boost). The first and second vaccines may be the same or different but are intended to both elicit an immune response useful in treating or preventing the same disease or condition. In some embodiments the prime is one or more viral proteins or portions thereof and the boost is one or more viral proteins or portions thereof.

The term “associated” or “associated with” as used herein to describe a disease (e.g., a virus associated disease or bacteria associated disease) means that the disease is caused by, or a symptom of the disease is caused by, what is described as disease associated or what is described as associated with the disease. As used herein, what is described as being associated with a disease, if a causative agent, could be a target for treatment of the disease.

The vaccine antigens described herein may be chemically coupled to a carrier or recombinantly expressed with an immunogenic carrier peptide or polypeptide (e.g., an antigen-carrier fusion peptide or polypeptide) to enhance an immune reaction. Means for conjugating a polypeptide or peptide to an immunogenic carrier protein are well known in the art and include, for example, glutaraldehyde, m-maleimidobenzoyl-N-hydroxysuccinimide ester, carbodiimide and bis-biazotized benzidine. In specific embodiments, the carrier is mutant SMEZ-2.

### A. SMEZ-2 Bacterial Superantigen

Streptococcal Mitogenic Exotoxin Z-2 (SMEZ-2) from Streptococcus pyogenes is a bacterial superantigen (SAg) that is the most immunogenic SAg discovered to date (Kamezawa et al., 1997; Proft et al., 2000). Wild-type SMEZ-2 binds with high affinity to both MHC class II molecules and T-cell receptors (TCRs), which indiscriminately activates T cells and can stimulate up to 20% of the body's T-cell pool (Li et al., 1999). This leads to the generation of a non-specific immune response that results in a massive release of cytokines and toxic shock syndrome at microgram quantities of SAgs (Alouf et al., 2003). The T cell binding and mitogenic (and toxic) effects of SMEZ-2 can be abolished by mutation of key residues (W75L, K182Q, and D42C) on the TCRVβ binding face, a modification that gives rise to a protein that maintains high affinity for MHC class II molecules without the toxic effects associated with wild-type SMEZ-2 (Radcliff et al., 2012). The MHC class II binding of mutant SMEZ-2 therefore offers an efficient carrier system for targeting antigens directly to antigen-presenting cells (APCs). This effectively “hijacks” the function of SMEZ-2 through its detoxification and results in a protein “carrier” for efficient presentation of conjugated antigens (Dickgreber et al., 2009).

### B. Target Proteins

Among the target proteins of antigens targeted by the present vaccine conjugates are those expressed in the context of a disease, condition, or cell type to be targeted via the vaccine conjugate. Among the diseases and conditions are proliferative, neoplastic, and malignant diseases and disorders, including cancers and tumors, including hematologic cancers, cancers of the immune system, such as lymphomas, leukemias, and/or myelomas, such as B. T, and myeloid leukemias, lymphomas, and multiple myelomas. In some embodiments, the antigen is selectively expressed or overexpressed on cells of the disease or condition, e.g., the tumor or pathogenic cells, as compared to normal or non-targeted cells or tissues. In other embodiments, the antigen is expressed on normal cells and/or is expressed on the engineered cells. Any suitable antigen may find use in the present methods. Exemplary antigens include, but are not limited to, antigenic molecules from infectious agents, auto-/self-antigens, tumor-/cancer-associated antigens, and tumor neoantigens.

The terms “tumor-associated antigen,” “tumor antigen” and “cancer cell antigen” are used interchangeably herein. In each case, the terms refer to proteins, glycoproteins or carbohydrates that are specifically or preferentially expressed by cancer cells.

A tumor associated antigen may be of any kind so long as it is expressed on the cell surface of tumor cells. Tumor-associated antigens may be derived from prostate, breast, colorectal, lung, pancreatic, renal, mesothelioma, ovarian, sarcoma or melanoma cancers. Exemplary tumor-associated antigens or tumor cell-derived antigens include MAGE 1, 3, and MAGE 4 (or other MAGE antigens such as those disclosed in International Patent Publication No. WO99/40188); PRAME; BAGE; RAGE, Lage (also known as NY ESO 1); SAGE; and HAGE or GAGE. These non-limiting examples of tumor antigens are expressed in a wide range of tumor types such as melanoma, lung carcinoma, sarcoma, and bladder carcinoma. See, e.g., U.S. Pat. No. 6,544,518. Prostate cancer tumor-associated antigens include, for example, prostate specific membrane antigen (PSMA), prostate-specific antigen (PSA), prostatic acid phosphates, NKX3.1, and six-transmembrane epithelial antigen of the prostate (STEAP).

Exemplary embodiments of tumor associated antigens include CD19, CD20, carcinoembryonic antigen, alphafetoprotein, CA-125, MUC-1, CD56, EGFR, c-Met, AKT, Her2, Her3, epithelial tumor antigen, melanoma-associated antigen, mutated p53, mutated ras, and so forth. In particular aspects, the antigens include NY-ESO, EGFRvIII, Muc-1, Her2, CA-125, WT-1, Mage-A3, Mage-A4, Mage-A10, TRAIL/DR4, and CEA. In particular aspects, the antigens for the two or more antigen receptors include, but are not limited to, CD19, EBNA, WT1, CD123, NY-ESO, EGFRvIII, MUC1, HER2, CA-125, WT1, Mage-A3, Mage-A4, Mage-A10, TRAIL/DR4, and/or CEA. The sequences for these antigens are known in the art, for example, CD19 (Accession No. NG_007275.1), EBNA (Accession No. NG_002392.2), WT1 (Accession No. NG_009272.1), CD123 (Accession No. NC_000023.11), NY-ESO (Accession No. NC_000023.11), EGFRvIII (Accession No. NG_007726.3), MUC1 (Accession No. NG_029383.1), HER2 (Accession No. NG_007503.1), CA-125 (Accession No. NG_055257.1), WT1 (Accession No. NG_009272.1), Mage-A3 (Accession No. NG_013244.1), Mage-A4 (Accession No. NG_013245.1), Mage-A10 (Accession No. NC_000023.11), TRAIL/DR4 (Accession No. NC_000003.12), and/or CEA (Accession No. NC_000019.10).

Other tumor associated antigens include Plu-1, HASH-1, HasH-2, Cripto and Criptin. Additionally, a tumor antigen may be a self-peptide hormone, such as whole length gonadotrophin hormone releasing hormone (GnRH), a short 10 amino acid long peptide, useful in the treatment of many cancers.

Tumor antigens include tumor antigens derived from cancers that are characterized by tumor-associated antigen expression, such as HER-2/neu expression. Tumor-associated antigens of interest include lineage-specific tumor antigens such as the melanocyte-melanoma lineage antigens MART-1/Melan-A, gp100, gp75, mda-7, tyrosinase and tyrosinase-related protein. Illustrative tumor-associated antigens include, but are not limited to, tumor antigens derived from or comprising any one or more of, p53, Ras, c-Myc, cytoplasmic serine/threonine kinases (e.g., A-Raf, B-Raf, and C-Raf, cyclin-dependent kinases), MAGE-A1, MAGE-A2, MAGE-A3, MAGE-A4, MAGE-A6, MAGE-A10, MAGE-A12, MART-1, BAGE, DAM-6, -10, GAGE-1, -2, -8, GAGE-3, -4, -5, -6, -7B, NA88-A, MART-1, MC1R, Gp100, PSA, PSM, Tyrosinase, TRP-1, TRP-2, ART-4, CAMEL, CEA, Cyp-B, hTERT, hTRT, iCE, MUC1, MUC2, Phosphoinositide 3-kinases (PI3Ks), TRK receptors, PRAME, P15, RU1, RU2, SART-1, SART-3, Wilms' tumor antigen (WT1), AFP, -catenin/m, Caspase-8/m, CEA, CDK-4/m, ELF2M, GnT-V, G250, HSP70-2M, HST-2, KIAA0205, MUM-1, MUM-2, MUM-3, Myosin/m, RAGE, SART-2, TRP-2/INT2, 707-AP, Annexin II. CDC27/m, TPI/mbcr-abl, BCR-ABL, interferon regulatory factor 4 (IRF4), ETV6/AML, LDLR/FUT, Pml/RAR, Tumor-associated calcium signal transducer 1 (TACSTD1) TACSTD2, receptor tyrosine kinases (e.g., Epidermal Growth Factor receptor (EGFR) (in particular, EGFRvIII), platelet derived growth factor receptor (PDGFR), vascular endothelial growth factor receptor (VEGFR)), cytoplasmic tyrosine kinases (e.g., src-family, syk-ZAP70 family), integrin-linked kinase (ILK), signal transducers and activators of transcription STAT3, STATS, and STATE, hypoxia inducible factors (e.g., HIF-1 and HIF-2). Nuclear Factor-Kappa B (NF-B), Notch receptors (e.g., Notch1-4), c-Met, mammalian targets of rapamycin (mTOR), WNT, extracellular signal-regulated kinases (ERKs), and their regulatory subunits, PMSA, PR-3, MDM2, Mesothelin, renal cell carcinoma-5T4, SM22-alpha, carbonic anhydrases I (CAI) and IX (CAIX) (also known as G250), STEAD, TEL/AML1, GD2, proteinase3, hTERT, sarcoma translocation breakpoints, EphA2, ML-IAP, EpCAM, ERG (TMPRSS2 ETS fusion gene), NA17, PAX3, ALK, androgen receptor, cyclin B1, polysialic acid, MYCN, RhoC, GD3, fucosyl GM1, mesothelian, PSCA, sLe, PLAC1, GM3, BORIS, Tn, GLoboH, NY-BR-1, RGsS, SART3, STn, PAX5, OY-TES1, sperm protein 17, LCK, HMWMAA, AKAP-4, SSX2, XAGE 1, B7H3, legumain, TIE2, Page4, MAD-CT-1, FAP, MAD-CT-2, fos related antigen 1, CBX2, CLDN6, SPANX, TPTE, ACTL8, ANKRD30A, CDKN2A, MAD2L1, CTAG1B, SUNC1, LRRN1 and idiotype.

Antigens may include epitopic regions or epitopic peptides derived from genes mutated in tumor cells or from genes transcribed at different levels in tumor cells compared to normal cells, such as telomerase enzyme, survivin, mesothelin, mutated ras, bcr/abl rearrangement, Her2/neu, mutated or wild-type p53, cytochrome P450 1B1, and abnormally expressed intron sequences such as N-acetylglucosaminyltransferase-V; clonal rearrangements of immunoglobulin genes generating unique idiotypes in myeloma and B-cell lymphomas; tumor antigens that include epitopic regions or epitopic peptides derived from oncoviral processes, such as human papilloma virus proteins E6 and E7; Epstein bar virus protein LMP2; nonmutated oncofetal proteins with a tumor-selective expression, such as carcinoembryonic antigen and alpha-fetoprotein.

In other embodiments, an antigen is obtained or derived from a pathogenic microorganism or from an opportunistic pathogenic microorganism (also called herein an infectious disease microorganism), such as a virus, fungus, parasite, and bacterium. In certain embodiments, antigens derived from such a microorganism include full-length proteins.

### C. Vaccine Conjugate Production

A variety of commercially available vectors and expression systems can be employed to generate the present vaccine conjugates, including those designed for mammalian cells, insect (Spodoptera; Baculovirus delivered) cells and bacterial cells. In some aspects, the purification is performed using E. coli, such as E. coli BL21(DE3) cells, HEK cells, or CHO cells.

In certain embodiments, the vaccine conjugate is purified. The term “purified,” as used herein, is intended to refer to a composition, isolatable from other components, wherein the protein is purified to any degree relative to its naturally obtainable state. A purified protein therefore also refers to a protein, free from the environment in which it may naturally occur. Where the term “substantially purified” is used, this designation will refer to a composition in which the protein or peptide forms the major component of the composition, such as constituting about 50%, about 60%, about 70%, about 80%, about 90%, about 95%, about 98%, about 99% or more of the proteins in the composition.

In certain embodiments, a plasmid vector is contemplated for use to transform a host cell. In general, plasmid vectors containing replicon and control sequences which are derived from species compatible with the host cell are used in connection with these hosts. The vector ordinarily carries a replication site, as well as marking sequences which are capable of providing phenotypic selection in transformed cells. In a non-limiting example, E. coli is often transformed using derivatives of pBR322, a plasmid derived from an E. coli species. pBR322 contains genes for ampicillin and tetracycline resistance and thus provides easy means for identifying transformed cells. The pBR plasmid, or other microbial plasmid or phage must also contain, or be modified to contain, for example, promoters which can be used by the microbial organism for expression of its own proteins. In some aspects, the vaccine conjugate is cloned into pET15TEV_NESG which expresses the protein with an N-terminal 6×-histidine tag that is cleavable by the Tobacco Etch Virus (TEV) protease. The vaccine conjugate is expressed in BL21(DE3) E. coli and purified using immobilized metal affinity chromatography IMAC. In order to avoid non-specific immunogenicity or unanticipated side effects, the 6×-histidine tag is excised by overnight digestion with TEV, purified by another round of IMAC where the flow-through contained purified protein.

In addition, phage vectors containing replicon and control sequences that are compatible with the host microorganism can be used as transforming vectors in connection with these hosts. For example, the phage lambda GEM™-11 may be utilized in making a recombinant phage vector which can be used to transform host cells, such as, for example, E. coli LE392.

Further useful plasmid vectors include pIN vectors (Inouye et al., 1985); and pGEX vectors, for use in generating glutathione S-transferase (GST) soluble fusion proteins for later purification and separation or cleavage. Other suitable fusion proteins are those with -galactosidase, ubiquitin, and the like.

Bacterial host cells, for example, E. coli, comprising the expression vector, are grown in any of a number of suitable media, for example, LB. The expression of the recombinant protein in certain vectors may be induced, as would be understood by those of skill in the art, by contacting a host cell with an agent specific for certain promoters, e.g., by adding IPTG to the media or by switching incubation to a higher temperature. After culturing the bacteria for a further period, generally of 2-24 hr, the cells are collected by centrifugation and washed to remove residual media.

Protein purification techniques are well known to those of skill in the art. These techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure peptide are ion-exchange chromatography, exclusion chromatography; polyacrylamide gel electrophoresis; isoelectric focusing. Other methods for protein purification include precipitation with ammonium sulfate, PEG, antibodies and the like or by heat denaturation, followed by centrifugation; gel filtration, reverse phase, hydroxylapatite and affinity chromatography; and combinations of such and other techniques. In purifying a protein, it may be desirable to extract the protein using denaturing conditions. The polypeptide may be purified from other cellular components using an affinity column, which binds to a tagged portion of the polypeptide. As is generally known in the art, it is believed that the order of conducting the various purification steps may be changed, or that certain steps may be omitted, and still result in a suitable method for the preparation of a substantially purified protein or peptide.

Various methods for quantifying the degree of purification of the protein or peptide will be known to those of skill in the art in light of the present disclosure. These include, for example, determining the specific activity of an active fraction, or assessing the amount of polypeptide within a fraction by SDS/PAGE analysis. Another method for assessing the purity of a fraction is to calculate the specific activity of the fraction, to compare it to the specific activity of the initial extract and to thus calculate the degree of purity. The actual units used to represent the amount of activity will, of course, be dependent upon the particular assay technique chosen to follow the purification and whether or not the expressed protein or peptide exhibits a detectable activity. It is known that the migration of a polypeptide can vary, sometimes significantly, with different conditions of SDS/PAGE (Capaldi et al., 1977). It will therefore be appreciated that under differing electrophoresis conditions, the apparent molecular weights of purified or partially purified expression products may vary.

One of skill in the art would be well-equipped to construct a vector through standard recombinant techniques (see, for example, Sambrook et al., 2001 and Ausubel et al., 1996, both incorporated herein by reference) for the expression of the antigen receptors of the present disclosure. Vectors include, but are not limited to, plasmids, cosmids, viruses (bacteriophage, animal viruses, and plant viruses), and artificial chromosomes (e.g., YACs), such as retroviral vectors (e.g., derived from Moloney murine leukemia virus vectors (MoMLV), MSCV, SFFV, MPSV, SNV etc.), lentiviral vectors (e.g., derived from HIV-1, HIV-2, SIV, BIV, FIV etc.), adenoviral (Ad) vectors including replication competent, replication deficient and gutless forms thereof, adeno-associated viral (AAV) vectors, simian virus 40 (SV-40) vectors, bovine papilloma virus vectors, Epstein-Barr virus vectors, herpes virus vectors, vaccinia virus vectors, Harvey murine sarcoma virus vectors, murine mammary tumor virus vectors, Rous sarcoma virus vectors, parvovirus vectors, polio virus vectors, vesicular stomatitis virus vectors, maraba virus vectors and group B adenovirus enadenotucirev vectors.

### 1. Regulatory Elements

Expression cassettes included in vectors useful in the present disclosure in particular contain (in a 5′-to-3′ direction) a eukaryotic transcriptional promoter operably linked to a protein-coding sequence, splice signals including intervening sequences, and a transcriptional termination/polyadenylation sequence. The promoters and enhancers that control the transcription of protein encoding genes in eukaryotic cells are composed of multiple genetic elements. The cellular machinery is able to gather and integrate the regulatory information conveyed by each element, allowing different genes to evolve distinct, often complex patterns of transcriptional regulation. A promoter used in the context of the present disclosure includes constitutive, inducible, and tissue-specific promoters.

### 2. Promoter/Enhancers

The expression constructs provided herein comprise a promoter to drive expression of the antigen receptor. A promoter generally comprises a sequence that functions to position the start site for RNA synthesis. The best-known example of this is the TATA box, but in some promoters lacking a TATA box, such as, for example, the promoter for the mammalian terminal deoxynucleotidyl transferase gene and the promoter for the SV40 late genes, a discrete element overlying the start site itself helps to fix the place of initiation. Additional promoter elements regulate the frequency of transcriptional initiation. Typically, these are located in the region 30110 bp-upstream of the start site, although a number of promoters have been shown to contain functional elements downstream of the start site as well. To bring a coding sequence “under the control of” a promoter, one positions the 5′ end of the transcription initiation site of the transcriptional reading frame “downstream” of (i.e., 3 of) the chosen promoter. The “upstream” promoter stimulates transcription of the DNA and promotes expression of the encoded RNA.

The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either cooperatively or independently to activate transcription. A promoter may or may not be used in conjunction with an “enhancer,” which refers to a cis-acting regulatory sequence involved in the transcriptional activation of a nucleic acid sequence.

A promoter may be one naturally associated with a nucleic acid sequence, as may be obtained by isolating the 5 non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other virus, or prokaryotic or eukaryotic cell, and promoters or enhancers not “naturally occurring,” i.e., containing different elements of different transcriptional regulatory regions, and/or mutations that alter expression. For example, promoters that are most commonly used in recombinant DNA construction include the lactamase (penicillinase), lactose and tryptophan (trp-) promoter systems. In addition to producing nucleic acid sequences of promoters and enhancers synthetically, sequences may be produced using recombinant cloning and/or nucleic acid amplification technology, including PCR™, in connection with the compositions disclosed herein. Furthermore, it is contemplated that the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the organelle, cell type, tissue, organ, or organism chosen for expression. Those of skill in the art of molecular biology generally know the use of promoters, enhancers, and cell type combinations for protein expression, (see, for example Sambrook et al. 1989, incorporated herein by reference). The promoters employed may be constitutive, tissue-specific, inducible, and/or useful under the appropriate conditions to direct high-level expression of the introduced DNA segment, such as is advantageous in the large-scale production of recombinant proteins and/or peptides. The promoter may be heterologous or endogenous.

Additionally, any promoter/enhancer combination (as per, for example, the Eukaryotic Promoter Data Base EPDB) could also be used to drive expression. Use of a T3, T7 or SP6 cytoplasmic expression system is another possible embodiment. Eukaryotic cells can support cytoplasmic transcription from certain bacterial promoters if the appropriate bacterial polymerase is provided, either as part of the delivery complex or as an additional genetic expression construct.

Non-limiting examples of promoters include early or late viral promoters, such as, SV40 early or late promoters, cytomegalovirus (CMV) immediate early promoters, Rous Sarcoma Virus (RSV) early promoters; eukaryotic cell promoters, such as, e.g., beta actin promoter, GADPH promoter, metallothionein promoter; and concatenated response element promoters, such as cyclic AMP response element promoters (cre), serum response element promoter (sre), phorbol ester promoter (TPA) and response element promoters (tre) near a minimal TATA box. It is also possible to use human growth hormone promoter sequences (e.g., the human growth hormone minimal promoter described at Genbank, accession no. X05244, nucleotide 283-341) or a mouse mammary tumor promoter (available from the ATCC, Cat. No. ATCC 45007). In certain embodiments, the promoter is CMV IE, dectin-1, dectin-2, human CD11c, F4/80, SM22, RSV, SV40, Ad MLP, beta-actin, MHC class I or MHC class II promoter, however any other promoter that is useful to drive expression of the therapeutic gene is applicable to the practice of the present disclosure.

In certain aspects, methods of the disclosure also concern enhancer sequences, i.e., nucleic acid sequences that increase a promoter's activity and that have the potential to act in cis, and regardless of their orientation, even over relatively long distances (up to several kilobases away from the target promoter). However, enhancer function is not necessarily restricted to such long distances as they may also function in close proximity to a given promoter.

### 3. Initiation Signals and Linked Expression

A specific initiation signal also may be used in the expression constructs provided in the present disclosure for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

In certain embodiments, the use of internal ribosome entry sites (IRES) elements are used to create multigene, or polycistronic, messages. IRES elements are able to bypass the ribosome scanning model of 5′ methylated Cap dependent translation and begin translation at internal sites. IRES elements from two members of the picornavirus family (polio and encephalomyocarditis) have been described, as well an IRES from a mammalian message. IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message.

Additionally, certain 2A sequence elements could be used to create linked- or co-expression of genes in the constructs provided in the present disclosure. For example, cleavage sequences could be used to co-express genes by linking open reading frames to form a single cistron. An exemplary cleavage sequence is the F2A (Foot-and-mouth disease virus 2A) or a “2A-like” sequence (e.g., Thosea asigna virus 2A; T2A).

### 4. Origins of Replication

In order to propagate a vector in a host cell, it may contain one or more origins of replication sites (often termed “ori”), for example, a nucleic acid sequence corresponding to oriP of EBV as described above or a genetically engineered oriP with a similar or elevated function in programming, which is a specific nucleic acid sequence at which replication is initiated. Alternatively, a replication origin of other extra-chromosomally replicating virus as described above or an autonomously replicating sequence (ARS) can be employed.

### 5. Selection and Screenable Markers

In some embodiments, cells containing a construct of the present disclosure may be identified in vitro or in vivo by including a marker in the expression vector. Such markers would confer an identifiable change to the cell permitting easy identification of cells containing the expression vector. Generally, a selection marker is one that confers a property that allows for selection. A positive selection marker is one in which the presence of the marker allows for its selection, while a negative selection marker is one in which its presence prevents its selection. An example of a positive selection marker is a drug resistance marker.

Usually, the inclusion of a drug selection marker aids in the cloning and identification of transformants, for example, genes that confer resistance to neomycin, puromycin, hygromycin, DHFR, GPT, zeocin and histidinol are useful selection markers. In addition to markers conferring a phenotype that allows for the discrimination of transformants based on the implementation of conditions, other types of markers including screenable markers such as GFP, whose basis is colorimetric analysis, are also contemplated. Alternatively, screenable enzymes as negative selection markers such as herpes simplex virus thymidine kinase (tk) or chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selection and screenable markers are well known to one of skill in the art.

### D. Formulation and Administration

The present disclosure provides pharmaceutical compositions comprising mutant SMEZ-2 conjugated to target protein(s). Such compositions comprise a prophylactically or therapeutically effective amount of a target protein or a fragment thereof, or a peptide immunogen, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a particular carrier when the pharmaceutical composition is administered intravenously. Saline solutions and aqueous dextrose and glycerol solutions can also be employed as liquid carriers, particularly for injectable solutions. Other suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

The composition, if desired, can also contain minor amounts of wetting or emulsifying agents, or pH buffering agents. These compositions can take the form of solutions, suspensions, emulsion, tablets, pills, capsules, powders, sustained-release formulations and the like. Oral formulations can include standard carriers such as pharmaceutical grades of mannitol, lactose, starch, magnesium stearate, sodium saccharine, cellulose, magnesium carbonate, etc. Examples of suitable pharmaceutical agents are described in “Remington's Pharmaceutical Sciences.” Such compositions will contain a prophylactically or therapeutically effective amount of the antibody or fragment thereof, preferably in purified form, together with a suitable amount of carrier so as to provide the form for proper administration to the patient. The formulation should suit the mode of administration, which can be oral, intravenous, intraarterial, intrabuccal, intranasal, nebulized, bronchial inhalation, or delivered by mechanical ventilation.

Active vaccines can be formulated for parenteral administration. e.g., formulated for injection via the intradermal, intravenous, intramuscular, subcutaneous, or even intraperitoneal routes. Administration by intradermal and intramuscular routes are contemplated. The vaccine could alternatively be administered by a topical route directly to the mucosa, for example by nasal drops, inhalation, or by nebulizer. Pharmaceutically acceptable salts, include the acid salts and those which are formed with inorganic acids such as, for example, hydrochloric or phosphoric acids, or such organic acids as acetic, oxalic, tartaric, mandelic, and the like. Salts formed with the free carboxyl groups may also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

Generally, the ingredients of compositions of the disclosure are supplied either separately or mixed together in unit dosage form, for example, as a dry lyophilized powder or water-free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compositions of the disclosure can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

As is also well known in the art, the immunogenicity of a particular immunogen composition can be enhanced by the use of non-specific stimulators of the immune response, known as adjuvants. Adjuvants have been used experimentally to promote a generalized increase in immunity against poorly immunogenic antigens (e.g., U.S. Pat. No. 4,877,611). Immunization protocols have used adjuvants to stimulate responses for many years, and as such adjuvants are well known to one of ordinary skill in the art. Some adjuvants affect the way in which antigens are presented. For example, the immune response is increased when protein antigens are adsorbed to alum. Emulsification of antigens also prolongs the duration of antigen presentation and initiates an innate immune response. Suitable molecule adjuvants include all acceptable immunostimulatory compounds, such as cytokines, toxins or synthetic compositions.

The term “adjuvant” is used in accordance with its plain ordinary meaning within Immunology and refers to a substance that is commonly used as a component of an immunogenic composition. Adjuvants may increase an antigen specific immune response in a subject when administered to the subject with one or more specific antigens as part of an immunogenic composition. In some embodiments, an adjuvant accelerates an immune response to an antigen. In some embodiments, an adjuvant prolongs an immune response to an antigen. In some embodiments, an adjuvant enhances an immune response to an antigen.

Those of skill in the art will know the different kinds of adjuvants that can be conjugated to vaccines in accordance with this disclosure and which are approved for human vs experimental use. These include alkyl lysophospholipids (ALP); BCG; and biotin (including biotinylated derivatives) among others. Certain adjuvants particularly contemplated for use are the teichoic acids from Gram bacterial cells. These include lipoteichoic acids (LTA), ribitol teichoic acids (RTA) and glycerol teichoic acid (GTA). Active forms of their synthetic counterparts may also be employed in connection with the compositions of this disclosure (Takada et al., 1995).

In some aspects, the compositions described herein may further comprise an adjuvant. Although Alum is an approved adjuvant for humans, adjuvants in experimental animals include complete Freund's adjuvant (a non-specific stimulator of the immune response containing killed Mycobacterium tuberculosis), incomplete Freund's adjuvants and aluminum hydroxide adjuvant. Other adjuvants that may also be used in animals and sometimes humans include Interleukin (IL)-1, IL-2, IL-4, TL-7, IL-12, interferon, Bacillus Calmette-Guérin (BCG), aluminum hydroxide, muramyl dipeptide (MDP) compounds, such as thur-MDP and nor-MDP (N-acctylmuramyl-L-alanyl-D-isoglutamine MDP), lipid A, and monophosphoryl lipid A (MPL). RIBI, which contains three components extracted from bacteria, MPL, trehalose dimycolate (TDM) and cell wall skeleton (CWS) in a 2% squalene/Tween 80 emulsion also is contemplated. MHC antigens may even be used.

### E. Methods of Treatment

In particular, the compositions that may be used in treating cancer in a subject (e.g., a human subject) are disclosed herein. The compositions described above are preferably administered to a mammal (e.g., rodent, human, non-human primates, canine, bovine, ovine, equine, feline, etc.) in an effective amount, that is, an amount capable of producing a desirable result in a treated subject (e.g., causing apoptosis of cancerous cells or killing bacterial cells). Toxicity and therapeutic efficacy of the compositions utilized in methods of the disclosure can be determined by standard pharmaceutical procedures. As is well known in the medical and veterinary arts, dosage for any one animal depends on many factors, including the subject's size, body surface area, body weight, age, the particular composition to be administered, time and route of administration, general health, the clinical symptoms of the infection or cancer and other drugs being administered concurrently. A composition as described herein is typically administered at a dosage that inhibits the growth or proliferation of a bacterial cell, inhibits the growth of a biofilm, or induces death of cancerous cells (e.g., induces apoptosis of a cancer cell), as assayed by identifying a reduction in hematological parameters (complete blood count (CBC)), or cancer cell growth or proliferation.

As used herein, the term “subject” refers to a human or any non-human animal (e.g., mouse, rat, rabbit, dog, cat, cattle, swine, sheep, horse or primate). A human includes pre- and post-natal forms. In many embodiments, a subject is a human being. A subject can be a patient, which refers to a human presenting to a medical provider for diagnosis or treatment of a disease. The term “subject” is used herein interchangeably with “individual” or “patient.” A subject can be afflicted with or is susceptible to a disease or disorder but may or may not display symptoms of the disease or disorder.

The term “therapeutically effective amount” or “effective dosage” as used herein refers to the dosage or concentration of a drug effective to treat a disease or condition. For example, with regard to the use of the monoclonal antibodies or antigen-binding fragments thereof disclosed herein to treat cancer, a therapeutically effective amount is the dosage or concentration of the monoclonal antibody or antigen-binding fragment thereof capable of reducing the tumor volume, eradicating all or part of a tumor, inhibiting or slowing tumor growth or cancer cell infiltration into other organs, inhibiting growth or proliferation of cells mediating a cancerous condition, inhibiting or slowing tumor cell metastasis, ameliorating any symptom or marker associated with a tumor or cancerous condition, preventing or delaying the development of a tumor or cancerous condition, or some combination thereof.

“Treating” or “treatment” of a condition as used herein includes preventing or alleviating a condition, slowing the onset or rate of development of a condition, reducing the risk of developing a condition, preventing or delaying the development of symptoms associated with a condition, reducing or ending symptoms associated with a condition, generating a complete or partial regression of a condition, curing a condition, or some combination thereof.

The therapeutic methods of the disclosure (which include prophylactic treatment) in general include administration of a therapeutically effective amount of the compositions described herein to a subject in need thereof, including a mammal, particularly a human. Such treatment will be suitably administered to subjects, particularly humans, suffering from, having, susceptible to, or at risk for a disease, disorder, or symptom thereof. Determination of those subjects “at risk” can be made by any objective or subjective determination by a diagnostic test or opinion of a subject or health care provider (e.g., genetic test, enzyme or protein marker, marker (as defined herein), family history, and the like).

In one embodiment, the disclosure provides a method of monitoring treatment progress. The method includes the step of determining a level of changes in hematological parameters and/or cancer stem cell (CSC) analysis with cell surface proteins as diagnostic markers (which can include, for example, but are not limited to CD34, CD38, CD90, and CD117) or diagnostic measurement (e.g., screen, assay) in a subject suffering from or susceptible to a disorder or symptoms thereof associated with cancer (e.g., leukemia) in which the subject has been administered a therapeutic amount of a composition as described herein. The level of marker determined in the method can be compared to known levels of marker either in healthy normal controls or in other afflicted patients to establish the subject's disease status. In preferred embodiments, a second level of marker in the subject is determined at a time point later than the determination of the first level, and the two levels are compared to monitor the course of disease or the efficacy of the therapy. In certain preferred embodiments, a pre-treatment level of marker in the subject is determined prior to beginning treatment according to the methods described herein; this pre-treatment level of marker can then be compared to the level of marker in the subject after the treatment commences, to determine the efficacy of the treatment.

### F. Combination Therapies

It is envisioned that vaccine conjugates described herein may be used in combination therapies with an additional anti-cancer agent, or a compound which mitigates one or more of the side effects of the disease or the therapy experienced by the patient. The following is a general discussion of therapies that may be used in conjunction with the therapies of the present disclosure. To treat cancers using the methods and compositions of the present disclosure, one would generally contact a tumor cell or subject with a composition of the present disclosure and at least one other therapy. These therapies would be provided in a combined amount effective to achieve a reduction in one or more disease parameter(s). This process may involve contacting the cells/subjects with both agents/therapies at the same time, e.g., using a single composition or pharmacological formulation that includes both agents, or by contacting the cell/subject with two distinct compositions or formulations, at the same time, wherein one composition includes the compound and the other includes the other agent. Alternatively, the compositions of the present disclosure may precede or follow the other treatment by intervals ranging from minutes to weeks. One would generally ensure that a significant period of time did not expire between the time of each delivery, such that the therapies would still be able to exert an advantageously combined effect on the cell/subject. In such instances, it is contemplated that one would contact the cell with both modalities within about 12-24 hours of each other, within about 6-12 hours of each other, or with a delay time of only about 12 hours. In some situations, it may be desirable to extend the time period for treatment significantly; however, where several days (2, 3, 4, 5, 6 or 7) to several weeks (1, 2, 3, 4, 5, 6, 7 or 8) lapse between the respective administrations.

It also is conceivable that more than one administration of either the compound or the other therapy will be desired. Various combinations may be employed, where a vaccine conjugate is “A,” and the other therapy is “B,” as exemplified below:

A/B/A B/A/B B/B/A A/A/B B/A/A A/B/B B/B/B/A B/B/A/B
A/A/B/B A/B/A/B A/B/B/A B/B/A/A B/A/B/A B/A/A/B B/B/B/A
A/A/A/B B/A/A/A A/B/A/A A/A/B/A A/B/B/B B/A/B/B B/B/A/B

The following are examples of standard anti-cancer therapies that could be used in combination with the compositions and methods of the present application.

### 1. Chemotherapy

The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis. Most chemotherapeutic agents fall into the following categories: alkylating agents, antimetabolites, antitumor antibiotics, mitotic inhibitors, and nitrosoureas.

Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin γ₁¹ and calicheamicin ω₁¹; dynemicin, including dynemicin A uncialamycin and derivatives thereof; bisphosphonates, such as clodronate; an esperamicin; as well as neocarzinostatin chromophore and related chromoprotein enediyne antiobiotic chromophores, aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel and doxetaxel; chlorambucil; gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids such as retinoic acid; capecitabine; cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, paclitaxel, docetaxel, gemcitabien, navelbine, farnesyl-protein transferase inhibitors, transplatinum, 5-fluorouracil, vincristin, vinblastin and methotrexate and pharmaceutically acceptable salts, acids or derivatives of any of the above.

### 2. Radiotherapy

Radiotherapy, also called radiation therapy, is the treatment of cancer and other diseases with ionizing radiation. Ionizing radiation deposits energy that injures or destroys cells in the area being treated by damaging their genetic material, making it impossible for these cells to continue to grow. Although radiation damages both cancer cells and normal cells, the latter are able to repair themselves and function properly.

Radiation therapy used according to the present disclosure may include, but is not limited to, the use of -rays, X-rays, and/or the directed delivery of radioisotopes to tumor cells. Other forms of DNA damaging factors are also contemplated such as microwaves and UV-irradiation. It is most likely that all of these factors induce a broad range of damage on DNA, on the precursors of DNA, on the replication and repair of DNA, and on the assembly and maintenance of chromosomes. Dosage ranges for X-rays range from daily doses of 12.9 to 51.6 mC/kg for prolonged periods of time (3 to 4 wk), to single doses of 0.516 to 1.55 C/kg. Dosage ranges for radioisotopes vary widely, and depend on the half-life of the isotope, the strength and type of radiation emitted, and the uptake by the neoplastic cells.

Radiotherapy may comprise the use of radiolabeled antibodies to deliver doses of radiation directly to the cancer site (radioimmunotherapy). Antibodies are highly specific proteins that are made by the body in response to the presence of antigens (substances recognized as foreign by the immune system). Some tumor cells contain specific antigens that trigger the production of tumor-specific antibodies. Large quantities of these antibodies can be made in the laboratory and attached to radioactive substances (a process known as radiolabeling). Once injected into the body, the antibodies actively seek out the cancer cells, which are destroyed by the cell-killing (cytotoxic) action of the radiation. This approach can minimize the risk of radiation damage to normal cells. Conformal radiotherapy uses the same radiotherapy machine, a linear accelerator, as the normal radiotherapy treatment but metal blocks are placed in the path of the x-ray beam to alter its shape to match that of the cancer. This ensures that a higher radiation dose is given to the tumor. Normal surrounding cells and nearby structures receive a lower dose of radiation, so the possibility of side effects is reduced. A device called a multi-leaf collimator has been developed and may be used as an alternative to metal blocks. The multi-leaf collimator consists of a number of metal sheets which are fixed to the linear accelerator. Each layer can be adjusted so that the radiotherapy beams can be shaped to the treatment area without the need for metal blocks. Precise positioning of the radiotherapy machine is very important for conformal radiotherapy treatment and a special scanning machine may be used to check the position of internal organs at the beginning of each treatment.

High-resolution intensity modulated radiotherapy also uses a multi-leaf collimator. During this treatment the layers of the multi-leaf collimator are moved while the treatment is being given. This method is likely to achieve even more precise shaping of the treatment beams and allows the dose of radiotherapy to be constant over the whole treatment area.

Although research studies have shown that conformal radiotherapy and intensity modulated radiotherapy may reduce the side effects of radiotherapy treatment, it is possible that shaping the treatment area so precisely could stop microscopic cancer cells just outside the treatment area being destroyed. This means that the risk of the cancer coming back in the future may be higher with these specialized radiotherapy techniques.

Scientists also are looking for ways to increase the effectiveness of radiation therapy. Two types of investigational drugs are being studied for their effect on cells undergoing radiation. Radiosensitizers make the tumor cells more likely to be damaged, and radioprotectors protect normal tissues from the effects of radiation. Hyperthermia, the use of heat, is also being studied for its effectiveness in sensitizing tissue to radiation.

### 3. Immunotherapy

In the context of cancer treatment, immunotherapeutics, generally, rely on the use of immune effector cells and molecules to target and destroy cancer cells. Trastuzumab (Herceptin™) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells. The combination of therapeutic modalities. i.e., direct cytotoxic activity and inhibition or reduction of ErbB2 would provide therapeutic benefit in the treatment of ErbB2 overexpressing cancers.

In one aspect of immunotherapy, the tumor cell must bear some marker that is amenable to targeting, i.e., is not present on the majority of other cells. Many tumor markers exist and any of these may be suitable for targeting in the context of the present disclosure. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155. An alternative aspect of immunotherapy is to combine anticancer effects with immune stimulatory effects. Immune stimulating molecules also exist including: cytokines such as IL-2, IL-4, IL-12, GM-CSF, -IFN, chemokines such as MIP-1, MCP-1, IL-8 and growth factors such as FLT3 ligand. Combining immune stimulating molecules, either as proteins or using gene delivery in combination with a tumor suppressor has been shown to enhance anti-tumor effects (Ju et al., 2000). Moreover, antibodies against any of these compounds may be used to target the anti-cancer agents discussed herein.

Examples of immunotherapies currently under investigation or in use are immune adjuvants e.g., Mycobacterium bovis, Plasmodium falciparum, dinitrochlorobenzene and aromatic compounds (U.S. Pat. Nos. 5,801,005 and 5,739,169), cytokine therapy, e.g., interferons α, and; IL-1, GM-CSF and TNF, gene therapy, e.g., TNF. IL-1, IL-2, p53 (U.S. Pat. Nos. 5,830,880 and 5,846,945) and monoclonal antibodies, e.g., anti-ganglioside GM2, anti-HER-2, anti-p185 (U.S. Pat. No. 5,824,311).

In active immunotherapy, an antigenic peptide, polypeptide or protein, or an autologous or allogenic tumor cell composition or “vaccine” is administered, generally with a distinct bacterial adjuvant (Ravindranath and Morton, 1991; Morton et al., 1992; Mitchell et al., 1990; Mitchell et al., 1993).

In adoptive immunotherapy, the patient's circulating lymphocytes, or tumor-infiltrated lymphocytes, are isolated in vitro, activated by lymphokines such as IL-2 or transduced with genes for tumor necrosis, and readministered.

Checkpoint inhibitors are an emerging class of immunotherapeutic. Checkpoint inhibitor therapy is a form of cancer immunotherapy currently under research. The therapy targets immune checkpoints, key regulators of the immune system that stimulate or inhibit its actions, which tumors can use to protect themselves from attacks by the immune system. Checkpoint therapy can block inhibitory checkpoints, restoring immune system function. The first anti-cancer drug targeting an immune checkpoint was ipilimumab, a CTLA4 blocker approved in the United States in 2011.

Currently approved checkpoint inhibitors target the molecules CTLA4, PD-1, and PD-L1, PD-1 is the transmembrane programmed cell death 1 protein (also called PDCD1 and CD279), which interacts with PD-L1 (PD-1 ligand 1, or CD274). PD-L1 on the cell surface binds to PD1 on an immune cell surface, which inhibits immune cell activity. Among PD-L1 functions is a key regulatory role on T cell activities. It appears that (cancer-mediated) upregulation of PD-L1 on the cell surface may inhibit T cells that might otherwise attack. Antibodies that bind to either PD-1 or PD-L1 and therefore block the interaction may allow the T-cells to attack the tumor.

The first checkpoint antibody approved by the FDA was ipilimumab, approved in 2011 for treatment of melanoma. It blocks the immune checkpoint molecule CTLA-4. Clinical trials have also shown some benefits of anti-CTLA-4 therapy on lung cancer or pancreatic cancer, specifically in combination with other drugs.

However, patients treated with check-point blockade (specifically CTLA-4 blocking antibodies), or a combination of check-point blocking antibodies, are at high risk of suffering from immune-related adverse events such as dermatologic, gastrointestinal, endocrine, or hepatic autoimmune reactions. These are most likely due to the breadth of the induced T-cell activation when anti-CTLA-4 antibodies are administered by injection in the blood stream.

Using a mouse model of bladder cancer, researchers have found that a local injection of a low dose anti-CTLA-4 in the tumor area had the same tumor inhibiting capacity as when the antibody was delivered in the blood. At the same time the levels of circulating antibodies were lower, suggesting that local administration of the anti-CTLA-4 therapy might result in fewer adverse events.

Initial clinical trial results with IgG4 PD1 antibody Nivolumab (under the brand name Opdivo and developed by Bristol-Myers Squibb) were published in 2010. It was approved in 2014. Nivolumab is approved to treat melanoma, lung cancer, kidney cancer, bladder cancer, head and neck cancer, and Hodgkin's lymphoma.

Pembrolizumab (brand name Keytruda) is another PD1 inhibitor that was approved by the FDA in 2014 and was the second checkpoint inhibitor approved in the United States. Keytruda is approved to treat melanoma and lung cancer and is produced by Merck.

Spartalizumab (PDR001) is a PD-1 inhibitor currently being developed by Novartis to treat both solid tumors and lymphomas. In May 2016, PD-L1 inhibitor atezolizumab was approved for treating bladder cancer. Other modes of enhancing adoptive immunotherapy include targeting so-called intrinsic checkpoint blockades, e.g., CISH.

Immunological adverse effects may be caused by checkpoint inhibitors. Altering checkpoint inhibition can have diverse effects on most organ systems of the body. The precise mechanism is unknown but differs in some respects based on the molecule targeted.

### 4. Surgery

Approximately 60% of persons with cancer will undergo surgery of some type, which includes preventative, diagnostic or staging, curative, and palliative surgery. Curative surgery is a cancer treatment that may be used in conjunction with other therapies, such as the treatment of the present disclosure, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and microscopically controlled surgery (Mohs' surgery). It is further contemplated that the present disclosure may be used in conjunction with removal of superficial cancers, precancers, or incidental amounts of normal tissue.

Upon excision of part or all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

In some particular embodiments, after removal of the tumor, an adjuvant treatment with a compound of the present disclosure is believed to be particularly efficacious in reducing the reoccurrence of the tumor. Additionally, the compounds of the present disclosure can also be used in a neoadjuvant setting.

### 5. Other Agents

It is contemplated that other agents may be used with the present disclosure. These additional agents include immunomodulatory agents, agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Immunomodulatory agents include tumor necrosis factor; interferon alpha, beta, and gamma; IL-2 and other cytokines; F42K and other cytokine analogs; or MIP-1, MIP-1β, MCP-1, RANTES, and other chemokines. It is further contemplated that the upregulation of cell surface receptors or their ligands such as Fas/Fas ligand, DR4 or DR5/TRAIL (Apo-2 ligand) would potentiate the apoptotic inducing abilities of the present disclosure by establishment of an autocrine or paracrine effect on hyperproliferative cells. Increased intercellular signaling by elevating the number of GAP junctions would increase the anti-hyperproliferative effects on the neighboring hyperproliferative cell population. In other embodiments, cytostatic or differentiation agents may be used in combination with the present disclosure to improve the anti-hyperproliferative efficacy of the treatments. Inhibitors of cell adhesion are contemplated to improve the efficacy of the present disclosure. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with the present disclosure to improve the treatment efficacy.

There have been many advances in the therapy of cancer following the introduction of cytotoxic chemotherapeutic drugs. However, one of the consequences of chemotherapy is the development/acquisition of drug-resistant phenotypes and the development of multiple drug resistance. The development of drug resistance remains a major obstacle in the treatment of such tumors and therefore, there is an obvious need for alternative approaches such as gene therapy. Another form of therapy for use in conjunction with chemotherapy, radiation therapy or biological therapy includes hyperthermia, which is a procedure in which a patient's tissue is exposed to high temperatures (up to 106° F.). External or internal heating devices may be involved in the application of local, regional, or whole-body hyperthermia. Local hyperthermia involves the application of heat to a small area, such as a tumor. Heat may be generated externally with high-frequency waves targeting a tumor from a device outside the body. Internal heat may involve a sterile probe, including thin, heated wires or hollow tubes filled with warm water, implanted microwave antennae, or radiofrequency electrodes.

A patient's organ or a limb is heated for regional therapy, which is accomplished using devices that produce high energy, such as magnets. Alternatively, some of the patient's blood may be removed and heated before being perfused into an area that will be internally heated. Whole-body heating may also be implemented in cases where cancer has spread throughout the body. Warm-water blankets, hot wax, inductive coils, and thermal chambers may be used for this purpose.

The skilled artisan is directed to “Remington's Pharmaceutical Sciences” 15th Edition. Chapter 33, in particular pages 624-652. Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

It also should be pointed out that any of the foregoing therapies may prove useful by themselves in treating cancer.

### II. KITS

In various aspects of the embodiments, a kit is envisioned containing therapeutic agents and/or other therapeutic and delivery agents. In some embodiments, the disclosure contemplates a kit for preparing and/or administering a vaccine composition of the embodiments. The kit may comprise one or more sealed vials containing any of the pharmaceutical compositions of the present embodiments. The kit may include, for example, vaccine conjugates as well as reagents to prepare, formulate, and/or administer the components of the embodiments or perform one or more steps of the inventive methods. In some embodiments, the kit may also comprise a suitable container, which is a container that will not react with components of the kit, such as an eppendorf tube, an assay plate, a syringe, a bottle, or a tube. The container may be made from sterilizable materials such as plastic or glass.

The kit may comprise one or more reagents for a biotechnology product or assay. The kit may further comprise regents for in vitro assays such as western blots, flow cytometry, immunoprecipitation, ELISA, or immunofluorescence.

The kit may further include an instruction sheet that outlines the procedural steps of the methods set forth herein, and will follow substantially the same procedures as described herein or are known to those of ordinary skill in the art. The instruction information may be in a computer readable media containing machine-readable instructions that, when executed using a computer, cause the display of a real or virtual procedure of delivering a pharmaceutically effective amount of a therapeutic agent.

### III. EXAMPLES

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1—Development of SMEZ-2 Vaccine Conjugates

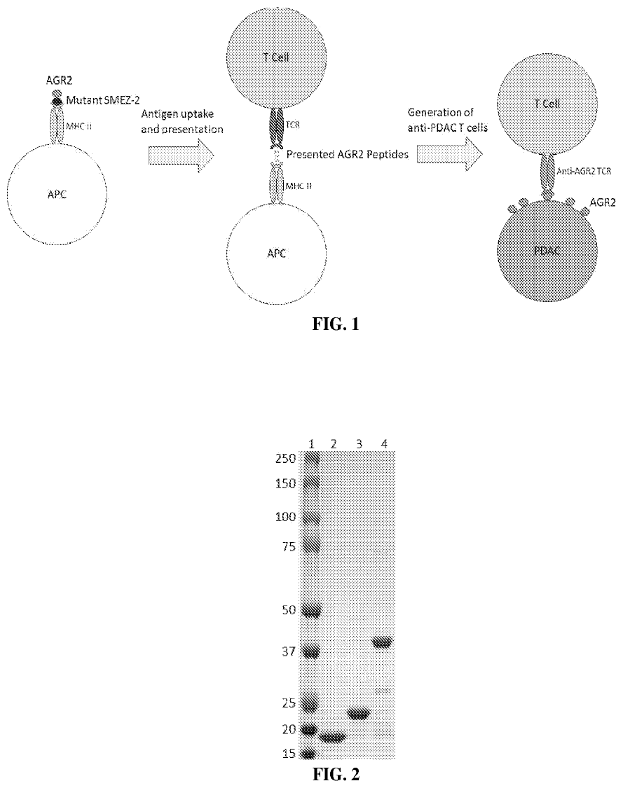

A detoxified AGR2-SMEZ-2 conjugate was developed that stimulates a robust immune response against PDAC with high extracellular AGR2. This conjugate harnesses the MHC-II binding abilities of SMEZ-2 to allow for efficient uptake and presentation of AGR2 peptides by APCs (FIG. 1). This technology overcomes current challenges presented by peptide-based cancer vaccines where in silico simulations with peptide binding to MHC molecules are no longer necessary due to the high affinity binding properties of SMEZ-2. Therefore, the present methods provide an AGR2-based PDAC vaccine, as well as other TAA and neoantigen-based vaccine candidates.

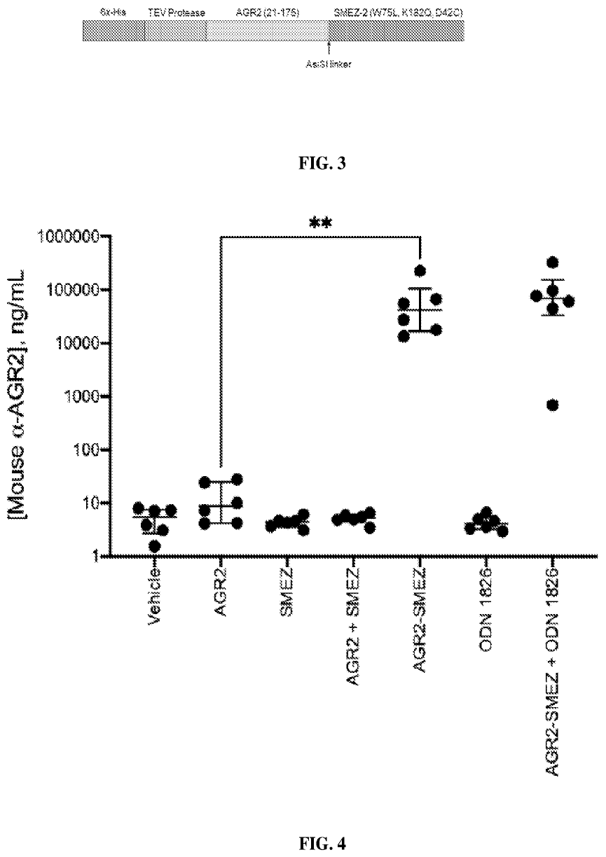

The candidate vaccine conjugate was synthesized by fusing the DNA sequence for human AGR2 (21-175) to the sequence of mutant SMEZ-2 (W75L, K182Q, D42C) that is de-toxified and lacks T-cell receptor binding using an AsiSI restriction site. The fusion was cloned into pET15TEV_NESG which expressed the protein with an N-terminal 6×-histidine tag that is cleavable by the Tobacco Etch Virus (TEV) protease (FIG. 3). The vaccine conjugate was expressed in BL21(DE3) E. coli and purified using immobilized metal affinity chromatography IMAC. In order to avoid non-specific immunogenicity or unanticipated side effects, the 6×-histidine tag was excised by overnight digestion with TEV, purified by another round of IMAC where the flow-through contained purified protein. The purity of the conjugate was determined by SDS-PAGE (>95%) (FIG. 2). AGR2 and SMEZ-2 were individually cloned, expressed, and purified using the same method as the conjugate. The proteins were very soluble in E. coli with yields of 33 mg/L of AGR2, 11 mg/L of SMEZ-2 (W75L, K182Q, D42C), and 4 mg/L of AGR2-SMEZ-2 (W75L, K182Q, D42C). As an alternative to expression in E. coli, these proteins may be produced in other cell types, such as HEK and CHO cells.

The immune responses elicited by unconjugated AGR2, unconjugated SMEZ2, the unconjugated versions of AGR2 and SMEZ mixed together, the AGR2-SMEZ2 conjugate, and the TLR9 agonist, CpG ODN1826, were tested in C57BL/6 mice by intramuscular injection in a PBS vehicle. CpG ODN1826 is a class B CpG oligonucleotide which contains a full phosphorothioate backbone with one or more CpG dinucleotides. Mice (n=10) were injected with 100 μL total volume with 100 μg AGR2-SMEZ-2 or equimolar amounts of the other proteins every two weeks. Anti-AGR2 specific IgG from the blood plasma of mice were analyzed by ELISA. Data in FIG. 4 were taken from blood samples 10 days after the second injection. The results show that 10 days after the second vaccination, anti-AGR2 antibody titers in the AGR2-SMEZ-2 treatment group were significantly greater than in mice treated with AGR2 alone or AGR2 injected simultaneously with SMEZ, suggesting the SMEZ2 carrier can enhance presentation of tumor associated antigens like AGR2 to the immune system.

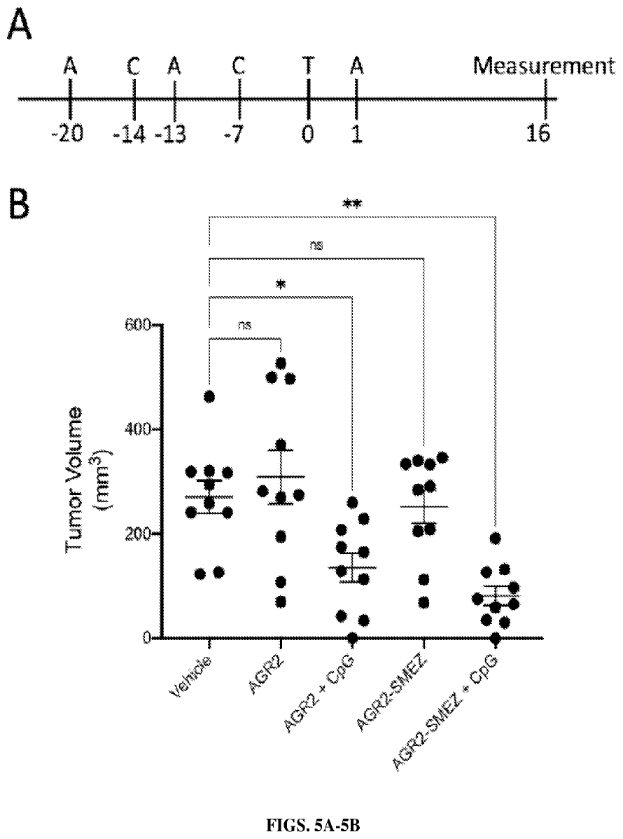

The anti-tumor potential of this therapeutic approach was next evaluated using a preclinical mouse model of pancreatic ductal adenocarcinoma (PDAC). Specifically, PDAC cells harvested from a pancreas tumor in LSL-KrasG12D; LSL-Trp53R172H; Pdx1-cre (KPC) mice were used. KPC32908 cells were engineered to express high levels of human AGR2, which has >95% sequence homology with mouse AGR2. Wild-type C57BL/6 mice (syngeneic with KPC mice) were then treated with the AGR2-SMEZ2 conjugate or appropriate controls with or without CpG ODN1826 according to the schedule shown in FIG. 5A. After two cycles of treatment, mice were challenged with KPC cells, which were injected subcutaneously into the rear flanks. This model rapidly develops aggressive KPC PDAC tumors that are resistant to standard of care chemotherapy and checkpoint inhibitor immunotherapies. However, the combination of AGR2-SMEZ2 and TLR9 agonist, CpG ODN1826, significantly reduced the growth rate of tumors (FIG. 5B), thereby demonstrating tumor control of pancreas tumors in a highly aggressive model system.

The activity of the SMEZ2 conjugate systems for other tumor associated antigens and tumor types/models were next evaluated. CD38 is actionable therapeutic target in multiple myeloma (MM), as demonstrated by the clinical efficacy of the anti-CD38 monoclonal antibody, daratumumab/DARZALEX, in newly diagnosed and relapsed/refractory MM patients [45-47]. To further demonstrate the potential of the SMEZ2 conjugate platform to enhance immune presentation of tumor associated antigens and induce a natural polyclonal humoral response, a mouse CD38-SMEZ2 conjugate was constructed, which was expressed and purified in a mammalian expression system. Purified protein products are shown in FIG. 6A. Wild-type BALB/C mice were then treated with two doses of 50 μg mCD38-SMEZ or equimolar equivalents of controls by subcutaneous administration in IFA. Blood plasma was taken 10 days after the second injection and CD38 specific IgG were quantified by ELISA. As shown in FIG. 6B, and consistent with data observed using the AGR2-SMEZ2 protein, a significantly higher level of CD38-specific IgG was measured in the blood of mice that received the CD38-SMEZ2 conjugate compared to cohorts that received CD38 alone or a mixture of free CD38 and SMEZ2. These results further demonstrate that the SMEZ2 superantigen platform has the ability to enhance presentation of multiple tumor associated antigens to the immune system.

The SMEZ2 platform was next evaluated against additional targets including immune cell localized antigens that regulate anti-tumor T cell responses. Specifically, constructs were created against Programmed cell Death protein-1 (PD-1, a.k.a. CD279) and Cytotoxic T Lymphocyte Associated protein-4 (CTLA-4, a.k.a. CD152), two inhibitory immune checkpoint molecules that suppress T cell function [35]. Antagonist antibodies, or immune checkpoint inhibitors (ICI's), directed against these molecules are clinically active in a range of tumor types, and the field has witnessed FDA approval of multiple ICI's including pembrolizumab/KETRUDA (anti-PD-1), nivolumab/OPDIVO (anti-PD-1), and ipilimumab/YERVOY (anti-CTLA-4) [36-40]. Monoclonal antibodies to Programmed Death Ligand-1 (PD-L1), which engages PD-1 to activate the PD-1 immune checkpoint and transduce suppressive T cell signals, have also shown utility in the clinic. Atezolizumab/TECENTRIQ was the first of the anti-PD-L1 antibodies to be approved by the FDA [41]. In addition to the role immune checkpoint molecules CTLA-4 and PD-1 play in suppressing anti-tumor T cell immunity, they are also highly expressed in malignant T cells, making them potential immunotherapy targets for lymphomas and leukemias that emerge from the T cell compartment. These include peripheral forms of T cell non-Hodgkins lymphoma such as cutaneous T cell lymphoma (CTCL), peripheral T cell lymphoma (PTCL), and NK/T cell lymphoma where expression of wild-type or gene fusions of PD-1 and CTLA-4 are expressed at high levels compared to normal T cells [42-44]. Therefore, immune checkpoints are actionable targets for driving an anti-tumor immune response in certain tumor types and may also be attractive therapeutic targets for T cell malignancies.

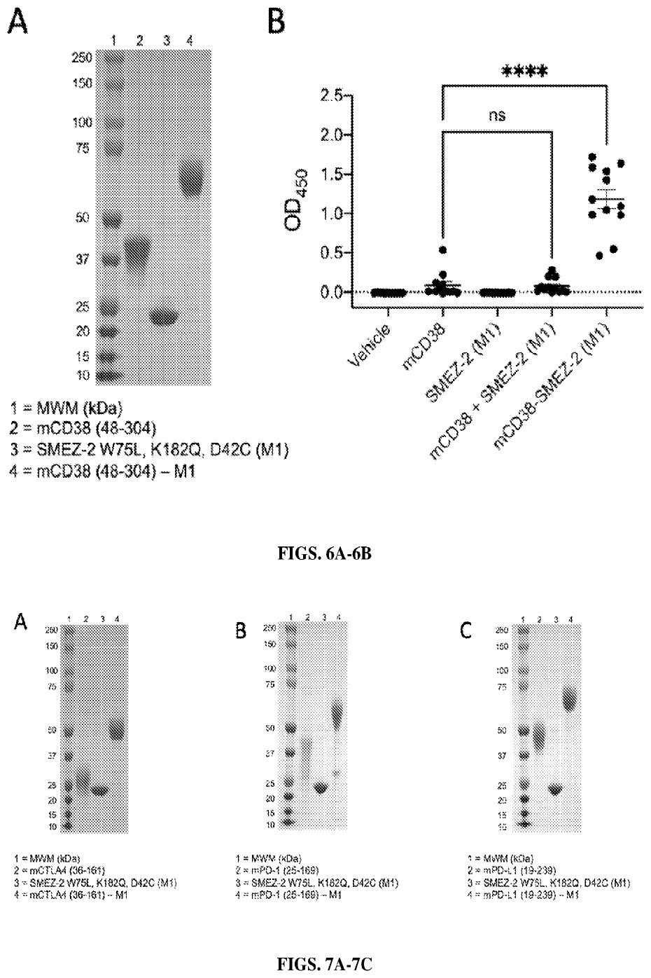

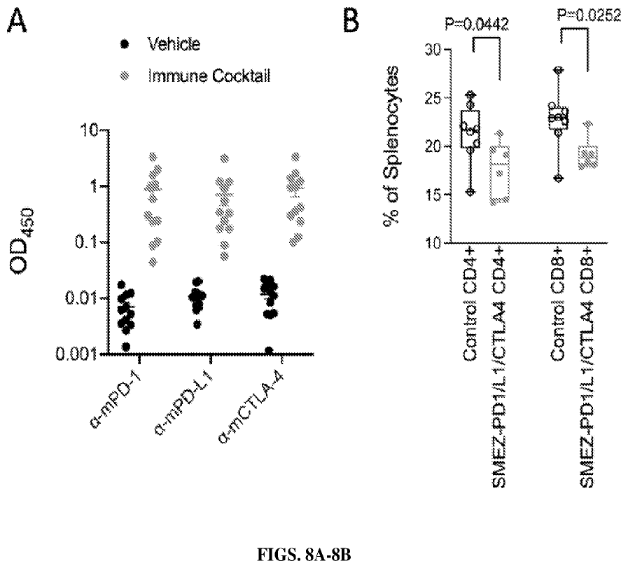

To explore the potential of targeting immune checkpoints using the SMEZ2 platform, mouse PD1-SMEZ, CTLA4-SMEZ, and PDL1-SMEZ constructs were created and expressed and purified in a mammalian cell culture system (FIG. 7). Next, the ability of the superantigen constructs to induce a PD-1/PD-L1/CTLA-4 specific immune response in vivo were evaluated. The results in FIG. 8 demonstrate that a cocktail containing all 3 conjugates was capable of inducing a strong polyclonal humoral response against mouse PD-1, PD-L1, and CTLA-4 (FIG. 8A). While it was hypothesized that antibodies generated against these antigens might lead to blockade of the immune checkpoints and increase T cell vitality, proliferation, and activity, the results demonstrate that the cocktails actually depleted T cell populations in the spleen (FIG. 8B)). This suggests that the response was predominantly an anti-T cell response rather than a T cell supporting effect. These results further prove the concept of the SMEZ platform and provide evidence of its versatility for multiple tumor and disease specific antigens. They further show that a SMEZ checkpoint construct or cocktail could be useful for depleting T cell populations in pathological situations such as T cell non-Hodgkin's lymphoma or autoimmune disorders.

### Example 2—Development of CD33-SMEZ-2 Vaccine Conjugate

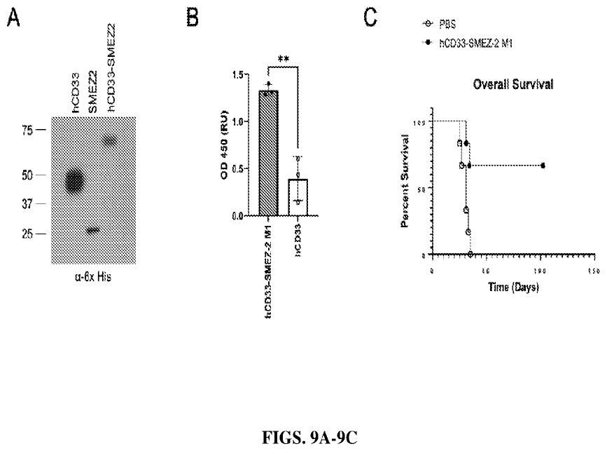

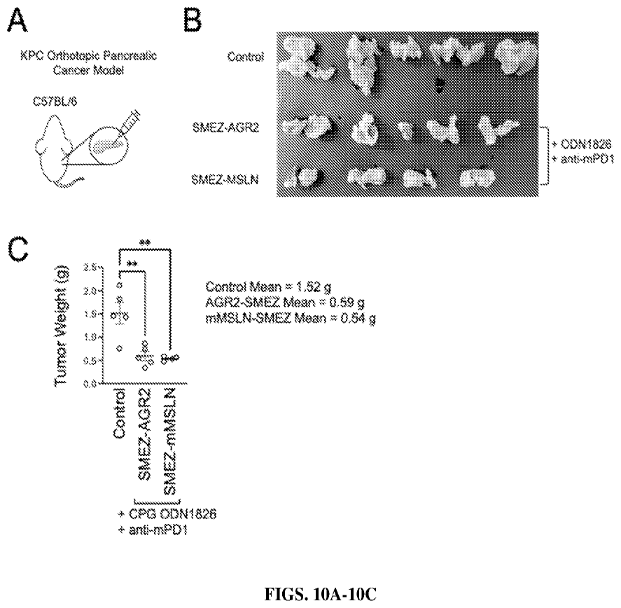

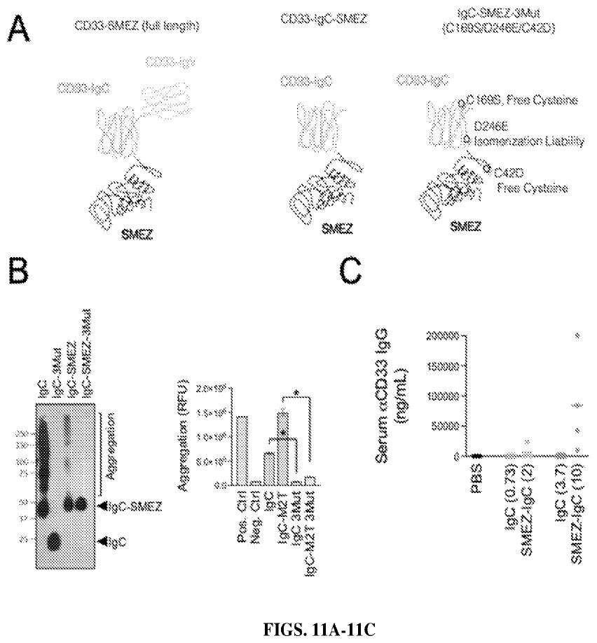

To evaluate the potential of a SMEZ-2 superantigen to drive an immune response against CD33+ AML, a full length human CD33-SMEZ-2 conjugate protein was cloned, produced, and purified in a mammalian cell culture system (FIG. 9A). The purified proteins were then tested in vivo using an immunocompetent syngeneic mouse model of AML in C57BL/6 mice. Proteins were injected subcutaneously in an emulsification of Freund's Incomplete Adjuvant (IFA).

At a dose of 40 pmoles injected at 2 week intervals, the CD33-SMEZ-2 conjugate was able to induce a significantly stronger humoral response against CD33 as determined by ELISA measurement of anti-hCD33 IgG in the blood plasma of inoculated mice (FIG. 9B).

To evaluate the anti-AML effects of the CD33-SMEZ-2 therapeutic, a syngeneic AML cell model of C1498 AML cells was used, which arose spontaneously in a C57BL/6 mouse. Mice were treated with two doses of CD33-SMEZ-2, and then two weeks later were injected intravenously with 1×10⁶ C1498 cells transduced with the human CD33 gene. This treatment protocol significantly prolonged median survival (p<0.05 N=5) and cured 60% of treated mice with “cure” being defined as survival past 100 days.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Alouf et al., Int J Med Microbiol. 2003; 292(7-8):429-40.
Arlen et al., Crit Rev Immunol. 2007; 27(5):451-62.
Bernhardt et al., Br J Cancer. 2006; 95(11):1474-82.
Bolouri et al., Nat Med. 2018; 24(1):103-12.
Conroy et al., N Engl J Med. 2011; 364(19):1817-25.
Conroy et al., N Engl J Med. 2018; 379(25):2395-406.
Deacy et al., Front Immunol. 2021; 12:731845.
Di Maro et al., Mol Cancer. 2014; 13:160.
Dickgreber et al., J Immunol. 2009; 182(3):1260-9.
Fessart et al., Elife. 2016; 5.
Gamis et al., J Clin Oncol. 2014; 32(27):3021-32.
Gilliam et al., Pancreas. 2012; 41(3):374-9.
Gjertsen et al., Lancet. 1995; 346(8987):1399-400.
Gjertsen et al., Int J Cancer. 2001; 92(3):441-50.
Gunturu et al., Ther Adv Med Oncol. 2013; 5(1):81-9.
Hammerstrom et al., Pharmacotherapy. 2011; 31(8):813-28.
Kamezawa et al., Infect Immun. 1997; 65(9):3828-33.
Kantoff et al., N Engl J Med. 2010; 363(5):411-22.
Ladha et al., Leuk Lymphoma. 2021; 62(2):257-63.
Lamba et al., J Clin Oncol. 2017; 35(23):2674-82.
Le et al., Clin Cancer Res. 2019; 25(18):5493-502.
Li et al., Annu Rev Immunol. 1999; 17:435-66.
Linenberger et al., Blood. 2001; 98(4):988-94.
Ma et al., Am J Transl Res. 2017; 9(2):507-19.
Melero et al., Nat Rev Clin Oncol. 2014; 11(9):509-24.
Melief et al., J Clin Invest. 2015; 125(9):3401-12.
Moidu et al., Heliyon. 2020; 6(9):e05000.
Morais et al., J Pediatr (Rio J). 2021; 97(2):204-10.
Nat Biotechnol. 2017; 35(2):97.
Oettle et al., JAMA. 2007; 297(3):267-77.
Oettle et al., JAMA. 2013; 310(14):1473-81.
Patel et al., J Mol Biol. 2013; 425(5):929-43.
Proft et al., J Exp Med. 2000; 191(10):1765-76.
Radcliff et al., Clin Vaccine Immunol. 2012; 19(4):574-86.
Romero et al., Sci Transl Med. 2016; 8(334):334ps9.
Rosenberg et al., Nat Med. 2004; 10(9):909-15.
Sahin et al., Nature. 2020; 585(7823):107-12.
Siegel et al., CA Cancer J Clin. 2019; 69(1):7-34.
Siegel et al., CA Cancer J Clin. 2022; 72(1):7-33.
Tsujikawa et al., Clin Cancer Res. 2020; 26(14):3578-88.
Wang et al., Cancer Res. 2008; 68(2):492-7.

What is claimed is:

1. A vaccine conjugate comprising mutant Streptococcal Mitogenic Exotoxin Z-2 (SMEZ-2) conjugated to at least one target protein or fragment thereof, wherein the at least one target protein is overexpressed in a cancer.

2. The conjugate of claim 1, wherein the mutant SMEZ-2 comprises mutations W75L and K182Q.

3. The conjugate of claim 1 or 2, wherein the mutant SMEZ-2 comprises mutations W75L, K182Q, and/or D42C.

4. The conjugate of any of claims 1-3, wherein the mutant SMEZ-2 comprises mutations W75L, K182Q, and D42C.

5. The conjugate of any of claims 1-4, further comprising a linker.

6. The conjugate of claim 5, wherein the linker is a peptide linker.

7. The conjugate of claim 6, wherein the peptide linker is a glycine-serine linker.

8. The conjugate of claim 6 or 7, wherein the peptide linker comprises the sequence AIA or GGGGS.

9. The conjugate of any of claims 1-4, wherein the at least one target protein is a neoantigen or tumor-associated antigen (TAA).

10. The conjugate of any of claims 1-4, wherein the at least one target protein is anterior gradient 2 (AGR2).

11. The conjugate of claim 10, wherein AGR2 is human AGR2.

12. The conjugate of any of claims 1-4, wherein the at least one target protein is an immune checkpoint protein.

13. The conjugate of claim 12, wherein the immune checkpoint protein is CTLA-4, PD-1, PD-L1, PD-L2, LAG-3, BTLA, B7H3, B7H4, TIM3, KIR, or A2aR.

14. The conjugate of claim 12, wherein the immune checkpoint protein is CTLA-4, PD-1, or PD-L1.

15. The conjugate of any of claims 1-4, wherein the at least one target protein is CD38.

16. The conjugate of any of claims 1-9, wherein the at least one target protein is CD38, PD-1, PD-L1, CTLA-4, human epidermal growth factor receptor 2 (HER2), prostate-specific membrane antigen (PSMA), melanoma-associated antigen 3 (MAGE-A3), NY-ESO-1, IL-8, or Growth/differentiation factor-15 (GDF-1).

17. The conjugate of any of claims 1-9, wherein the at least one target protein is CD33 or fragment thereof, mesothelin (MSLN), B-cell maturation antigen (BCMA), GPRC5D, CD123, CLL-1 (CD371), CD19, CD30, or CD20.

18. The conjugate of claim 17, wherein the CD33 or fragment thereof comprises one or more amino acid substitutions.

19. The conjugate of claim 18, wherein the one or more amino acid substitutions are at D231, D246, C154 and/or C169.

20. The conjugate of claim 18, wherein the one or more amino acid substitutions are at D231E, D246E, C154S and/or C169S.

21. The conjugate of claim 18 or 19, wherein the CD33 consists of the CD33-IgC domain.

22. The conjugate of any of claims 1-16, wherein the vaccine conjugate comprises at least a second target protein or fragment thereof.

23. A pharmaceutical composition comprising a vaccine conjugate of any of claims 1-22 and an adjuvant.

24. The composition of claim 23, wherein the adjuvant is Incomplete Freund's Adjuvant.

25. The composition of claim 24, wherein the conjugate is formulated as a 1:1 emulsification with Incomplete Freund's Adjuvant (IFA).

26. The composition of claim 23, wherein the conjugate is formulated for intravenous infusion.

27. An expression vector comprising a sequence encoding mutant SMEZ-2 fused to a sequence encoding a target protein or fragment thereof.

28. The vector of claim 27, wherein the vector encodes a vaccine conjugate of any of claims 1-22.

29. A host cell comprising an expression vector of claim 27 or 28.

30. The host cell of claim 29, wherein the host cell is E. coli, human embryonic kidney cell (HEK293), or Chinese hamster ovary (CHO) cell.

31. A method for stimulating an immune response in a subject, comprising administering to the subject an effective amount of a vaccine conjugate of any of claims 1-22 or a pharmaceutical composition of any of claims 23-25.

32. The method of claim 31, wherein the immune response is an anti-cancer immune response.

33. The method of claim 31 or 32, wherein the subject has a cancer.

34. The method of claim 33, wherein the cancer is oral cancer, oropharyngeal cancer, nasopharyngeal cancer, respiratory cancer, urogenital cancer, gastrointestinal cancer, central or peripheral nervous system tissue cancer, an endocrine or neuroendocrine cancer or hematopoietic cancer, glioma, sarcoma, carcinoma, lymphoma, melanoma, fibroma, meningioma, brain cancer, oropharyngeal cancer, nasopharyngeal cancer, renal cancer, biliary cancer, pheochromocytoma, pancreatic islet cell cancer, Li-Fraumeni tumors, thyroid cancer, parathyroid cancer, pituitary tumors, adrenal gland tumors, osteogenic sarcoma tumors, multiple neuroendocrine type I and type II tumors, breast cancer, lung cancer, head and neck cancer, prostate cancer, esophageal cancer, tracheal cancer, liver cancer, bladder cancer, stomach cancer, pancreatic cancer, ovarian cancer, uterine cancer, cervical cancer, testicular cancer, colon cancer, rectal cancer or skin cancer.

35. The method of claim 33, wherein the cancer is pancreatic cancer.

36. The method of claim 35, wherein the pancreatic cancer is pancreatic ductal adenocarcinoma (PDAC).

37. The method of claim 33, wherein the cancer is breast cancer.

38. The method of claim 37, wherein the target protein is CD38 and the cancer is multiple myeloma.

39. The method of claim 37, wherein the cancer is lymphoma.

40. The method of claim 39, wherein the lymphoma is T cell non-Hodgkin's lymphoma.

41. The method of any of claims 31-37, wherein the immune response is an anti-AGR2 specific immune response.

42. The method of claim 41, wherein the anti-AGR2 specific immune response is detected by measuring an increased titer of AGR2 specific immunoglobulins in a sample of said subject's blood.

43. The method of any of claims 31-42, wherein the conjugate is administered by injection.

44. The method of any of claims 31-43, further comprising administering a second anti-cancer therapy to said subject.

45. The method of claim 44, wherein the second anti-cancer therapy is immunotherapy, chemotherapy, radiotherapy, gene therapy, surgery, hormonal therapy, anti-angiogenic therapy or cytokine therapy.

46. The method of claim 44, wherein the second anti-cancer therapy is immunotherapy.

47. The method of claim 46, wherein the immunotherapy is an immune checkpoint inhibitor.

48. The method of claim 47, wherein the immune checkpoint inhibitor is selected from an inhibitor of CTLA-4, PD-1, PD-L1, PD-L2, LAG-3, BTLA, B7H3, B7H4, TIM3, KIR, or A2aR.

49. The method of claim 47, wherein the immune checkpoint inhibitor comprises an anti-PD1 agent.

50. The method of claim 49, wherein the anti-PD1 agent comprises an anti-PD1 antibody, anti-PDL1 antibody, or an anti-PDL2 antibody.

51. The method of claim 49, wherein the anti-PD1 agent is nivolumab, pembrolizumab, pidilizumab, KEYTRUDA®, AMP-514, REGN2810, CT-011, BMS 936559, MPDL328OA or AMP-224.

52. The method of claim 47, wherein the immune checkpoint inhibitor is an anti-CTLA-4 antibody.

53. The method of claim 52, wherein the anti-CTLA-4 antibody is tremelimumab, YERVOY®, or ipilimumab.

54. A method of treating a subject having a cancer comprising administering a vaccine conjugate of any of claims 1-22 or a pharmaceutical composition of any of claims 23-25 to the subject.

55. The method of claim 54, wherein the cancer is oral cancer, oropharyngeal cancer, nasopharyngeal cancer, respiratory cancer, urogenital cancer, gastrointestinal cancer, central or peripheral nervous system tissue cancer, an endocrine or neuroendocrine cancer or hematopoietic cancer, glioma, sarcoma, carcinoma, lymphoma, melanoma, fibroma, meningioma, brain cancer, oropharyngeal cancer, nasopharyngeal cancer, renal cancer, biliary cancer, pheochromocytoma, pancreatic islet cell cancer, Li-Fraumeni tumors, thyroid cancer, parathyroid cancer, pituitary tumors, adrenal gland tumors, osteogenic sarcoma tumors, multiple neuroendocrine type I and type II tumors, breast cancer, lung cancer, head and neck cancer, prostate cancer, esophageal cancer, tracheal cancer, liver cancer, bladder cancer, stomach cancer, pancreatic cancer, ovarian cancer, uterine cancer, cervical cancer, testicular cancer, colon cancer, rectal cancer or skin cancer.

56. The method of claim 54, wherein the cancer is prostate cancer.

57. The method of claim 56, wherein the prostate cancer is pancreatic ductal adenocarcinoma (PDAC).

58. The method of claim 54, wherein the cancer is breast cancer.

59. The method of claim 54, wherein the target protein is CD38 and the cancer is multiple myeloma.

60. The method of claim 54, wherein the cancer is lymphoma.

61. The method of claim 60, wherein the lymphoma is T cell non-Hodgkin's lymphoma.

62. The method of any of claims 54-58, further comprising administering at least one immune checkpoint inhibitor to the subject.

63. The method of claim 62, wherein the administering at least one immune checkpoint inhibitor to the subject comprises administering the at least one immune checkpoint inhibitor before the vaccine conjugate.

64. The method of claim 62, wherein the administering at least one immune checkpoint inhibitor to the subject comprises administering the at least one immune checkpoint inhibitor after or concomitantly with the vaccine conjugate.

65. The method of any of claims 62-64, wherein the at least one immune checkpoint inhibitor is selected from an inhibitor of CTLA-4, PD-1, PD-L1, PD-L2, LAG-3, BTLA, B7H3, B7H4, TIM3, KIR, or A2aR.

66. The method of any of claims 62-65, wherein the at least one immune checkpoint inhibitor comprises an anti-PD1 agent.

67. The method of claim 66, wherein the anti-PD1 agent comprises an anti-PD1 antibody, anti-PDL1 antibody, or an anti-PDL2 antibody.

68. The method of claim 66, wherein the anti-PD1 agent is nivolumab, pembrolizumab, pidilizumab, KEYTRUDA®, AMP-514, REGN2810, CT-011, BMS 936559, MPDL328OA or AMP-224.

69. The method of any of claims 62-68, wherein the at least one immune checkpoint inhibitor is an anti-CTLA-4 antibody.

70. The method of claim 69, wherein the anti-CTLA-4 antibody is tremelimumab, YERVOY®, or ipilimumab.

71. The method of any of claims 62-70, wherein the subject is administered two immune checkpoint inhibitors.

72. The method of claim 71, wherein the two immune checkpoint inhibitors are an anti-PD1 antibody and an anti-CTL4 antibody.

73. The method of any of claims 54-72, wherein the vaccine conjugate is administered two or more times.

74. The method of any of claims 54-73, further comprising administering a further anti-cancer therapy to the subject.

75. The method of claim 74, wherein the further anti-cancer therapy is chemotherapy, radiotherapy, gene therapy, surgery, hormonal therapy, anti-angiogenic therapy or cytokine therapy.

76. The method of claim 74, wherein the further anti-cancer therapy comprises a TLR9 agonist and/or CD40 agonist.

77. The method of claim 76, wherein the TLR9 agonist is CpG ODN1826.

78. The method of claim 76, wherein the CD40 agonist is a CD40 agonistic antibody.

79. A kit comprising a vaccine conjugate of any of claims 1-22 or a pharmaceutical composition of any of claims 23-25.

80. The kit of claim 79, further comprising an immune checkpoint inhibitor.

81. The kit of claim 80, wherein the immune checkpoint inhibitor is an anti-PD1 antibody or CTLA-4 antibody.

82. The kit of any of claims 79-81, further comprising a TLR9 agonist.

83. The kit of claim 82, wherein the TLR9 agonist is CpG ODN1826.

84. A composition comprising a vaccine conjugate of any of claims 1-22 or a pharmaceutical composition of any of claims 23-25 for use in treating a cancer.

85. The composition of claim 84, further comprising an immune checkpoint inhibitor.

86. The composition of claim 85, wherein the immune checkpoint inhibitor is an anti-PD1 antibody or CTLA-4 antibody.

87. The composition of any of claims 84-86, further comprising a TLR9 agonist.

88. The composition of claim 82, wherein the TLR9 agonist is CpG ODN1826.

89. A vaccine conjugate comprising mutant Streptococcal Mitogenic Exotoxin Z-2 (SMEZ-2) conjugated to CD33.

90. The conjugate of claim 89, wherein the mutant SMEZ-2 comprises mutations W75L and K182Q.

91. The conjugate of claim 89 or 90, wherein the mutant SMEZ-2 comprises mutations W75L, K182Q, and/or D42C.

92. The conjugate of any of claims 89-91, wherein the mutant SMEZ-2 comprises mutations W75L, K182Q, and D42C.

93. The conjugate of claim 89, wherein CD33 is human CD33.

94. The conjugate of claim 89, wherein the CD33 or fragment thereof comprises one or more amino acid substitutions.

95. The conjugate of claim 94, wherein the one or more amino acid substitutions are at D231, D246, C154 and/or C169.

96. The conjugate of claim 94, wherein the one or more amino acid substitutions are D231E, D246E, C154S and/or C169S.

97. The conjugate of claim 94 or 95, wherein the CD33 consists of the CD33-IgC domain.

98. The conjugate of any of claims 89-93, wherein the conjugate further comprises a linker.

99. The conjugate of claim 98, wherein the linker is a peptide linker.

100. The conjugate of claim 99, wherein the peptide linker is a glycine serine linker.

101. The conjugate of claim 100, wherein the glycine serine linker is GGGGS.

102. The conjugate of any of claims 89-104, wherein the vaccine conjugate further comprises a target protein overexpressed in a cancer or fragment thereof.

103. A pharmaceutical composition comprising a vaccine conjugate of any of claims 89-102 and an adjuvant.

104. The composition of claim 103, wherein the adjuvant is Incomplete Freund's Adjuvant.

105. The composition of claim 104, wherein the conjugate is formulated as a 1:1 emulsification with Incomplete Freund's Adjuvant (IFA).

106. The composition of claim 103, wherein the conjugate is formulated for intravenous infusion.

107. An expression vector comprising a sequence encoding mutant SMEZ-2 fused to CD33 or fragment thereof.

108. The vector of claim 107, wherein the vector encodes a vaccine conjugate of any of claims 89-102.

109. A host cell comprising an expression vector of claim 107 or 108.

110. The host cell of claim 109, wherein the host cell is E. coli, human embryonic kidney cell (HEK293), or Chinese hamster ovary (CHO) cell.

111. A method for stimulating an immune response in a subject, comprising administering to the subject an effective amount of a vaccine conjugate of any of claims 89-102 or a pharmaceutical composition of any of claims 103-105.

112. The method of claim 111, wherein the immune response is an anti-cancer immune response.

113. The method of claim 111 or 112, wherein the subject has cancer.

114. The method of claim 113, wherein the cancer is oral cancer, oropharyngeal cancer, nasopharyngeal cancer, respiratory cancer, urogenital cancer, gastrointestinal cancer, central or peripheral nervous system tissue cancer, an endocrine or neuroendocrine cancer or hematopoietic cancer, glioma, sarcoma, carcinoma, lymphoma, melanoma, fibroma, meningioma, brain cancer, oropharyngeal cancer, nasopharyngeal cancer, renal cancer, biliary cancer, pheochromocytoma, pancreatic islet cell cancer, Li-Fraumeni tumors, thyroid cancer, parathyroid cancer, pituitary tumors, adrenal gland tumors, osteogenic sarcoma tumors, multiple neuroendocrine type I and type II tumors, breast cancer, lung cancer, head and neck cancer, prostate cancer, esophageal cancer, tracheal cancer, liver cancer, bladder cancer, stomach cancer, pancreatic cancer, ovarian cancer, uterine cancer, cervical cancer, testicular cancer, colon cancer, rectal cancer or skin cancer.

115. The method of claim 113, wherein the cancer is leukemia.

116. The method of claim 115, wherein the leukemia is acute myelogenous leukemia (AML).

117. The method of any of claims 111-120, wherein the immune response is an anti-CD33 specific immune response.

118. The method of claim 117, wherein the anti-CD33 specific immune response is detected by measuring an increased titer of CD33 specific immunoglobulins in a sample of said subject's blood.

119. The method of any of claims 111-118, wherein the conjugate is administered by injection.

120. The method of any of claims 111-119, further comprising administering a second anti-cancer therapy to said subject.

121. The method of claim 120, wherein the second anti-cancer therapy is immunotherapy, chemotherapy, radiotherapy, gene therapy, surgery, hormonal therapy, anti-angiogenic therapy or cytokine therapy.

122. The method of claim 120, wherein the second anti-cancer therapy comprises a TLR9 agonist.

123. The method of claim 122, wherein the TLR9 agonist is CpG ODN1826.

124. The method of claim 120, wherein the second anti-cancer therapy is immunotherapy.

125. The method of claim 124, wherein the immunotherapy is an immune checkpoint inhibitor.

126. The method of claim 125, wherein the immune checkpoint inhibitor is selected from an inhibitor of CTLA-4, PD-1, PD-L1, PD-L2, LAG-3, BTLA, B7H3, B7H4, TIM3, KIR, or A2aR.

127. The method of claim 125, wherein the immune checkpoint inhibitor comprises an anti-PD1 agent.

128. The method of claim 127, wherein the anti-PD1 agent comprises an anti-PD1 antibody, anti-PDL1 antibody, or an anti-PDL2 antibody.

129. The method of claim 127, wherein the anti-PD1 agent is nivolumab, pembrolizumab, pidilizumab, KEYTRUDA®, AMP-514, REGN2810, CT-011, BMS 936559, MPDL328OA or AMP-224.

130. The method of claim 125, wherein the immune checkpoint inhibitor is an anti-CTLA-4 antibody.

131. The method of claim 130, wherein the anti-CTLA-4 antibody is tremelimumab, YERVOY®, or ipilimumab.

132. A method of treating a subject having a cancer comprising administering a vaccine conjugate of any of claims 89-102 or a pharmaceutical composition of any of claims 103-105 to the subject.

133. The method of claim 132, wherein the cancer is oral cancer, oropharyngeal cancer, nasopharyngeal cancer, respiratory cancer, urogenital cancer, gastrointestinal cancer, central or peripheral nervous system tissue cancer, an endocrine or neuroendocrine cancer or hematopoietic cancer, glioma, sarcoma, carcinoma, lymphoma, melanoma, fibroma, meningioma, brain cancer, oropharyngeal cancer, nasopharyngeal cancer, renal cancer, biliary cancer, pheochromocytoma, pancreatic islet cell cancer, Li-Fraumeni tumors, thyroid cancer, parathyroid cancer, pituitary tumors, adrenal gland tumors, osteogenic sarcoma tumors, multiple neuroendocrine type I and type II tumors, breast cancer, lung cancer, head and neck cancer, prostate cancer, esophageal cancer, tracheal cancer, liver cancer, bladder cancer, stomach cancer, pancreatic cancer, ovarian cancer, uterine cancer, cervical cancer, testicular cancer, colon cancer, rectal cancer or skin cancer.

134. The method of claim 132, wherein the cancer is leukemia.

135. The method of claim 134, wherein the cancer is acute myelogenous leukemia (AML).

136. The method of claim 132, wherein the cancer is pancreatic cancer or breast cancer.

137. The method of any of claims 132-136, further comprising administering at least one immune checkpoint inhibitor to the subject.

138. The method of claim 137, wherein the administering at least one immune checkpoint inhibitor to the subject comprises administering the at least one immune checkpoint inhibitor before the vaccine conjugate.

139. The method of claim 137, wherein the administering at least one immune checkpoint inhibitor to the subject comprises administering the at least one immune checkpoint inhibitor after or concomitantly with the vaccine conjugate.

140. The method of any of claims 137-139, wherein the at least one immune checkpoint inhibitor is selected from an inhibitor of CTLA-4, PD-1, PD-L1, PD-L2, LAG-3, BTLA, B7H3, B7H4, TIM3, KIR, or A2aR.

141. The method of any of claims 137-140, wherein the at least one immune checkpoint inhibitor comprises an anti-PD1 agent.

142. The method of claim 141, wherein the anti-PD1 agent comprises an anti-PD1 antibody, anti-PDL1 antibody, or an anti-PDL2 antibody.

143. The method of claim 141, wherein the anti-PD1 agent is nivolumab, pembrolizumab, pidilizumab, KEYTRUDA®, AMP-514, REGN2810, CT-011, BMS 936559, MPDL328OA or AMP-224.

144. The method of any of claims 137-143, wherein the at least one immune checkpoint inhibitor is an anti-CTLA-4 antibody.

145. The method of claim 144, wherein the anti-CTLA-4 antibody is tremelimumab, YERVOY®, or ipilimumab.

146. The method of any of claims 137-145, wherein the subject is administered two immune checkpoint inhibitors.

147. The method of claim 146, wherein the two immune checkpoint inhibitors are an anti-PD1 antibody and an anti-CTL4 antibody.

148. The method of any of claims 132-147, wherein the vaccine conjugate is administered two or more times.

149. The method of any of claims 132-148, further comprising administering a further anti-cancer therapy to the subject.

150. The method of claim 149, wherein the further anti-cancer therapy is chemotherapy, radiotherapy, gene therapy, surgery, hormonal therapy, anti-angiogenic therapy or cytokine therapy.

151. The method of claim 149, wherein the further anti-cancer therapy comprises a TLR9 agonist.

152. The method of claim 151, wherein the TLR9 agonist is CpG ODN1826.

153. A kit comprising a vaccine conjugate of any of claims 89-102 or a pharmaceutical composition of any of claims 103-105.

154. The kit of claim 153, further comprising an immune checkpoint inhibitor.

155. The kit of claim 154, wherein the immune checkpoint inhibitor is an anti-PD1 antibody or CTLA-4 antibody.

156. The kit of claim 153, further comprising a TLR9 agonist.

157. The kit of claim 156, wherein the TLR9 agonist is CpG ODN1826.

158. A composition comprising a vaccine conjugate of any of claims 89-102 or a pharmaceutical composition of any of claims 103-105 for use in treating a cancer.

159. The composition of claim 158, further comprising an immune checkpoint inhibitor.

160. The composition of claim 158 or 159, further comprising a TLR9 agonist and/or CD40 agonist.
